(19) United States
(12) Patent Application Publication
(10) Pub. No.: US 2026/0092118 A1
(43) Pub. Date: Apr. 2, 2026

(54) TREATMENT OF CANCER USING AN ANTI-HLA-G/ANTI-CD3 BISPECIFIC ANTIBODY AND A 4-1BB (CD137) AGONIST

(71) Applicant: Hoffmann-La Roche Inc., Little Falls, NJ (US)

(72) Inventors: Meher MAJETY, Basel (CH); Carina HAGE, Penzberg (DE); Johannes SAM, Baden (CH)

(73) Assignee: Hoffmann-La Roche Inc., Little Falls, NJ (US)

(21) Appl. No.: 19/325,175

(22) Filed: Sep. 10, 2025

Related U.S. Application Data
(63) Continuation of application No. PCT/EP2024/056421, filed on Mar. 11, 2024.

(30) Foreign Application Priority Data
Mar. 13, 2023 (EP) 23161500.6

(51) Int. Cl. C07K 16/28; A61K 38/00; A61K 39/00; A61P 35/00; C07K 14/47; C07K 16/40

(52) U.S. Cl. CPC C07K 16/2833; A61P 35/00; C07K 14/4705; C07K 16/2809; C07K 16/40; A61K 38/00; A61K 2039/505; C07K 2317/31; C07K 2317/33; C07K 2317/522; C07K 2317/55; C07K 2317/565; C07K 2317/71; C07K 2319/00

(57) ABSTRACT

The present invention relates to the treatment of cancer, in particular to the treatment of cancer using an anti-HLA-G/anti-CD3 bispecific antibody and a 4-1BB (CD137) agonist.

### CROSS-REFERENCE TO RELATED APPLICATIONS

This application is a continuation of International Patent Application Serial No. PCT/EP2024/056421, filed Mar. 11, 2024, which claims the benefit of priority to EP Application Serial No. 23161500.6, filed Mar. 13, 2023, all of which are incorporated herein by reference in their entirety and for all purposes.

### SEQUENCE LISTING

This application contains a Sequence Listing which has been submitted electronically in XML format and is hereby incorporated by reference in its entirety. Said XML copy, created on Sep. 9, 2025, is named “P38324-US-1-SEQUENCE_LISTING.xml” and is 67,682 bytes in size.

### FIELD OF THE INVENTION

The present invention relates to the treatment of cancer, in particular to the treatment of HLA-G expressing cancer, using an anti-HLA-G/anti-CD3 bispecific antibody and a 4-1BB (CD137) agonist.

### BACKGROUND

T cell activating bispecific antibodies are a promising class of cancer therapeutics, designed to engage cytotoxic T cells against tumor cells. The simultaneous binding of such an antibody to CD3 on T cells and to an antigen expressed on the tumor cells will force a temporary interaction between tumor cell and T cell, causing activation of the T cell and subsequent lysis of the tumor cell.

The human major histocompatibility complex, class I, 6, also known as human leukocyte antigen G (HLA-G), is a protein that is encoded in humans by the HLA-G gene. HLA-G belongs to the HLA nonclassical class I heavy chain paralogues. This class I molecule is a heterodimer consisting of a heavy chain and a light chain (beta-2 microglobulin). The heavy chain is anchored in the membrane but can also be shedded/secreted. HLA-G is predominantly expressed on cytotrophoblasts in the placenta. Several tumors (including pancreatic, breast, skin, colorectal, gastric & ovarian) express HLA-G (Lin, A. et al., Mol Med. 21 (2015) 782-791; Amiot, L., et al., Cell Mol Life Sci. 68 (2011) 417-431). The expression has also been reported to be associated with pathological conditions like inflammatory diseases, GvHD and cancer. Expression of HLA-G has been reported to be associated with poor prognosis in cancer. Tumor cells escape host immune surveillance by inducing immune tolerance/suppression via HLA-G expression.

HLA-G shares high homology (>98%) with other MHC I molecules, therefore truly HLA-G specific antibodies with no crossreactivity to other MHC I molecules are difficult to generate. Due to the high polymorphism and high homology of the HLA family, most of the antibodies lack either truly specific HLA-G binding properties and often also bind or crossreact with other HLA family members (either as MHC complex with β2M or in its β2M-free form) or they simply do not inhibit binding of HLA-Gβ2M MHC complex to its receptors ILT2 and/or ILT4 (and are regarded as non-antagonistic antibodies). Antibodies specifically binding to HLA-G are described in WO 2019/202040, WO 2019/202041, WO 2022/24024 and WO 2022/129120. These documents also describe T cell bispecific antibodies (TCBs) comprising a binding moiety specifically binding to HLA-G.

Anti-tumor activity of HLA-G TCB has been demonstrated in vitro using different HLA-G positive tumor cell lines, and in vivo, using mouse models. T cell activation, IFNγ secretion, and cytotoxicity induced by HLA-G TCB were shown to be dose dependent and correlating to the HLA-G density on cell surface and the percentage of HLA-G positive cells. In line with the mode of action of TCBs, tumor growth inhibition was accompanied by increased cytokine secretion (including IFNγ), tumoral T cell infiltration and activation as reflected by increased expression of T cell activation markers including CD69, CD25 and Granzyme B. However, the activation and cytokine secretion by T cells is transient reverting to baseline after treatment. Furthermore, activated T cells can become exhausted and undergo activation-induced cell death. Therefore, to achieve complete elimination of tumor cells, there is a need for additional agents that preserve sustained T cell activation.

Co-stimulation of T cells through 4-1BB receptor (e.g. by 4-1BBL binding) leads to activation of multiple signaling cascades with in T cells, powerfully augmenting T cell activation. In combination with T cell receptor activating agents, agonistic 4-1BB antibodies enhance T cell proliferation, stimulate cytokine secretion, and decrease sensitivity of T cells to activation induced cell death.

In order to maximize the therapeutic benefit of HLA-G-targeting T cell activating antibodies, particularly in solid tumors, it would thus be desirable to enhance their effect.

### BRIEF DESCRIPTION OF THE INVENTION

The present invention enhances T cell responses (e.g. in solid tumor cancers) by a combination treatment of an HLA-G targeted T cell activating bispecific antibody with a 4-1BB (CD137) agonist providing a positive costimulatory signal (4-1BBL) to T cells, in particular a 4-1BB (CD137) agonist targeting a stromal antigen such as Fibroblast-activation protein (FAP). Fibroblast activation protein (FAP) is highly expressed by cancer-associated fibroblasts (CAFs) and has the ability to modulate the tumor microenvironment (TME) by remodeling the extracellular matrix (ECM). As described, FAP is upregulated on cancer-associated fibroblasts in the tumor, and the FAP specificity of the 4-1BB (CD137) agonist therefore provides T cell co-stimulation tightly restricted to the tumor.

The present inventors have found that a combination of HLA-G-targeted T cell activating bispecific antibodies with a 4-1BB (CD137) agonist leads to enhanced activity in HLA-G-expressing cancers as compared to HLA-G-targeted T cell activated bispecific antibody alone.

Using in vivo data from patient-derived tumor models, the inventors have surprisingly found that tumor cell lysis induced by anti-HLA-G/anti-CD3 bispecific antibody in the presence of FAP-expressing fibroblast cells was enhanced by the addition of the 4-1BB agonist FAP4-1BBL, even in patient-derived tumor models and/or at doses where the anti-HLA-G/anti-CD3 bispecific antibody alone showed little or no activity.

Accordingly, in a first aspect, the present invention provides an anti-HLA-G/anti-CD3 bispecific antibody for use in the treatment of a cancer in an individual, wherein the treatment comprises administration of the anti-HLA-G/anti-CD3 bispecific antibody in combination with a 4-1BB (CD137) agonist.

In a further aspect, the present invention provides a 4-1BB (CD137) agonist for use in the treatment of a cancer in an individual, wherein the treatment comprises administration of the 4-1BB (CD137) agonist in combination with an anti-HLA-G/anti-CD3 bispecific antibody.

In one aspect, the invention provides the use of an anti-HLA-G/anti-CD3 bispecific antibody in the manufacture of a medicament for the treatment of cancer in an individual, wherein the treatment comprises administration of the anti-HLA-G/anti-CD3 bispecific antibody in combination with a 4-1BB (CD137) agonist.

In a further aspect, the invention provides the use of a 4-1BB (CD137) agonist in the manufacture of a medicament for the treatment of cancer in an individual, wherein the treatment comprises administration of the 4-1BB (CD137) agonist in combination with an anti-HLA-G/anti-CD3 bispecific antibody.

In still a further aspect, the invention provides a method for treating cancer in an individual comprising administering to the individual an anti-HLA-G/anti-CD3 bispecific antibody and a 4-1BB (CD137) agonist.

In one aspect, the invention also provides a kit comprising a first medicament comprising an anti-HLA-G/anti-CD3 bispecific antibody and a second medicament comprising a 4-1BB (CD137) agonist, and optionally further comprising a package insert comprising instructions for administration of the first medicament in combination with the second medicament for treating cancer in an individual.

The anti-HLA-G/anti-CD3 bispecific antibodies, 4-1BB (CD137) agonists, methods, uses or kits described above and herein, may incorporate, singly or in combination, any of the features described in the following (unless the context dictates otherwise).

The anti-HLA-G/anti-CD3 bispecific antibody herein is a bispecific antibody that specifically binds to CD3 and to HLA-G, particularly to HLA-G on JEG3 cells (ATCC No. HTB36). In particular, useful anti-HLA-G/anti-CD3 bispecific antibodies for use in the present invention inhibit ILT2 binding to (HLA-G expressed on) JEG3 cells (ATCC No. HTB36) and/or bind to (HLA-G expressed on) JEG3 cells (ATCC No. HTB36) and inhibit ILT2 binding to (HLA-G expressed on) JEG-3 cells (ATCC No. HTB36). Particularly useful anti-HLA-G/anti-CD3 bispecific antibodies for use in the present invention are described e.g. in PCT publication no. WO 2022/129120 (incorporated herein by reference in its entirety).

### DETAILED DESCRIPTION OF THE INVENTION

### Definitions

The term “bispecific” means that the antibody is able to specifically bind to at least two distinct antigenic determinants. Typically, a bispecific antibody comprises two antigen-binding sites, each of which is specific for a different antigenic determinant. In certain aspects, the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen-binding moiety binds, forming an antigen-binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the terms “antigen-binding moiety” or “antigen-binding domain” are used interchangeably and refer to a polypeptide molecule that specifically binds to an antigenic determinant. In one aspect, an antigen-binding moiety is able to direct the entity to which it is attached (e.g. a second antigen-binding moiety) to a target site, for example to a specific type of tumor cell bearing the antigenic determinant. In another aspect, an antigen-binding moiety is able to activate signaling through its target antigen, for example a T cell receptor complex antigen. Antigen-binding moieties include antibodies and fragments thereof as further defined herein. Particular antigen-binding moieties include an antigen-binding domain of an antibody, comprising an antibody heavy chain variable region and an antibody light chain variable region. In certain aspects, the antigen-binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

As used herein, the term “specific binding” means that the binding is selective for the antigen and can be discriminated from unwanted or non-specific interactions. The ability of an antigen-binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one aspect, the extent of binding of an antigen-binding moiety to an unrelated protein is less than about 10% of the binding of the antigen-binding moiety to the antigen as measured, e.g., by SPR. In certain aspects, an antigen-binding moiety that binds to the antigen, or an antibody comprising that antigen-binding moiety, has a dissociation constant (K_{D}) of ≤1μ, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen-binding moiety and an antigen, or a receptor and its ligand). The affinity of a molecule X for its partner Y can generally be represented by the dissociation constant (K_{D})), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 which results from processing in the cell. The term also encompasses naturally occurring variants of CD3, e.g., splice variants or allelic variants. In one aspect, CD3 is human CD3, particularly the epsilon subunit of human CD3 (CD38). The amino acid sequence of human CD38 is shown in UniProt (www.uniprot.org) accession no. P07766 (entry version 217), or NCBI (www.ncbi.nlm.nih.gov/) RefSeq NP_000724.1. See also SEQ ID NO: 44. The amino acid sequence of cynomolgus [Macaca fascicularis] CD3ε is shown in NCBI GenBank no. BAB71849.1. See also SEQ ID NO: 45.

When used herein, the term “HLA-G”, “human HLA-G”, or “HLAG”, refers to the HLA-G human major histocompatibility complex, class I, G, also known as human leukocyte antigen G (HLA-G) (exemplary SEQ ID NO: 41). Typically, HLA-G forms a MHC class I complex together with β2 microglobulin (B2M or β2m). In one embodiment HLA-G refers to the MHC class I complex of HLA-G and β2 microglobulin. In one preferred embodiment HLA-G refers to the cell surface bound MHC class I complex of HLA-G and β2 microglobulin, also known as HLA-G1 (see e.g. WO 2022/129120 A1, FIG. 1, and Blaschitz et al., Molecular Human Reproduction, 11 (2005) 699-710, inter alia FIG. 1)

As used herein, an antibody (either mono-, multi- or bispecific) or antigen-binding moiety “binding to human HLA-G”, “specifically binding to human HLA-G”, “that binds to human HLA-G” or “anti-HLA-G” refers to an antibody/antigen-binding moiety specifically binding to the human HLA-G antigen or its extracellular domain (ECD) with a binding affinity of a KD-value of 5.0×10⁻⁸ mol/l or lower, in one embodiment of a KD-value of 1.0×10⁻⁹ mol/l or lower, in one embodiment of a KD-value of 5.0×10⁻⁸ mol/l to 1.0×10⁻¹³ mol/l. In one embodiment the antibody binds to HLA-G β2M MHC I complex comprising SEQ ID NO: 42.

The binding affinity is determined with a standard binding assay, such as surface plasmon resonance technique (BIAcore®, GE-Healthcare Uppsala, Sweden) e.g. using constructs comprising HLA-G extracellular domain (e.g. in its natural occurring three-dimensional structure). In one embodiment, binding affinity is determined with a standard binding assay using exemplary soluble HLA-G comprising MHC class I complex comprising SEQ ID NO: 42.

HLA-G has the regular MHC I fold and consists of two chains: Chain 1 consists of three domains: alpha 1, alpha 2 and alpha 3. The alpha 1 and alpha 2 domains form a peptide binding groove flanked by two alpha helices. Small peptides (approximately 9mers) can bind to this groove akin to other MHCI proteins. Chain 2 is beta 2 microglobulin (β2M) which is shared with various other MHCI proteins.

HLA-G can form functionally active complex oligomeric structures (Kuroki, K et al. Eur J Immunol. 37 (2007) 1727-1729). Disulfide-linked dimers are formed between Cys 42 of two HLA-G molecules. (Shiroishi M et al., J Biol Chem 281 (2006) 10439-10447. Trimers and Tetrameric complexes have also been described e.g. in Kuroki, K et al. Eur J Immunol. 37 (2007) 1727-1729, Allan D. S., et al. J Immunol Methods. 268 (2002) 43-50 and T Gonen-Gross et al., J Immunol 171 (2003) 1343-1351). HLA-G has several free cysteine residues, unlike most of the other MHC class I molecules. Boyson et al., Proc Nat Acad Sci USA, 99:16180 (2002) reported that the recombinant soluble form of HLA-G5 could form a disulfide-linked dimer with the intermolecular Cys42-Cys42 disulfide bond. In addition, the membrane-bound form of HLA-G1 can also form a disulfide-linked dimer on the cell surface of the JEG3 cell line, which endogenously expresses HLA-G. Disulfide-linked dimer forms of HLA-G1 and HLA-G5 have been found on the cell surface of trophoblast cells as well (Apps, R., Tissue Antigens, 68:359 (2006)).

HLA-G is predominantly expressed on cytotrophoblasts in the placenta. Several tumors (including pancreatic, breast, skin, colorectal, gastric & ovarian) express HLA-G (Lin, A. et al., Mol Med. 21 (2015) 782-791; Amiot, L., et al., Cell Mol Life Sci. 68 (2011) 417-431). The expression has also been reported to be associated with pathological conditions like inflammatory diseases, GvHD and cancer. Expression of HLA-G has been reported to be associated with poor prognosis in cancer. Tumor cells escape host immune surveillance by inducing immune tolerance/suppression via HLA-G expression.

For HLA-G, 7 isoforms exist, whereof 3 are secreted and 4 are membrane bound forms. The most important functional isoforms of HLA-G include beta-2 microglobulin (β2M)-associated HLA-G1 and HLA-G5. However, the tolerogenic immunological effect of these isoforms is different and is dependent on the form (monomer, dimer) of ligands and the affinity of the ligand-receptor interaction.

HLA-G protein can be produced using standard molecular biology techniques. The nucleic acid sequence for HLA-G isoforms is known in the art. See for example GenBank Accession No. AY359818.

The HLA-G isomeric forms promote signal transduction through ILTs (Ig-like transcripts), in particular ILT2, ILT4, or a combination thereof.

ILTs represent Ig types of activating and inhibitory receptors that are involved in regulation of immune cell activation and control the function of immune cells (Borges, L., et al., Curr Top Microbial Immunol, 244:123-136 (1999)). ILTs are categorized into three groups: (i) inhibitory, i.e. those containing a cytoplasmic immunoreceptor tyrosine-based inhibitory motif (ITIM) and transducing an inhibitory signal (ILT2, ILT3, ILT4, ILT5, and LIR8); (ii) activating, i.e. those containing a short cytoplasmic tail and a charged amino acid residue in the transmembrane domain (ILT1, ILT7, ILT8, and LIR6alpha) and delivering an activating signal through the cytoplasmic immunoreceptor tyrosine-based activating motif (ITAM) of the associated common gamma chain of Fc receptor; and (iii) the soluble molecule ILT6 which lacks the transmembrane domain. A number of recent studies have highlighted immunoregulatory roles for ILTs on the surface of antigen presenting cells (APC). ILT2, ILT3, and ILT4 receptors, the most characterized immune inhibitory receptors, are expressed on a wide range of immune cells including monocytes, B cells, dendritic cells, plasmacytoid dendritic cells and a subset of NK and T cells. ILT2 is expressed on T cells subsets and has been shown to inhibit activation and proliferation of these cells upon ligation (Colonna M. et al., J Immunol. 20011, 66:2514-2521, J Immunol 2000; 165:3742-3755). ILT3 and ILT4 are upregulated by exposing immature DC to known immunosuppressive factors, including IL-10, vitamin D3, or suppressor CD8 T cells (Chang, C. C., et al., Nat Immunol, 3:237-243 (2002)). The expression of ILTs on DC is tightly controlled by inflammatory stimuli, cytokines, and growth factors, and is down-regulated following DC activation (Ju, X. S., et al., Gene, 331:159-164 (2004)). The expression of ILT2 and ILT4 receptors is highly regulated by histone acetylation, which contributes to strictly controlled gene expression exclusively in the myeloid lineage of cells (Nakajima, H., J Immunol, 171:6611-6620 (2003)).

Engagement of the inhibitory receptors ILT2 and ILT4 alters the cytokine and chemokine secretion/release profile of monocytes and can inhibit Fc receptor signaling (Colonna, M., et al. J Leukoc Biol, 66:375-381 (1999)). The role and function of ILT3 on DC have been precisely described by the Suciu-Foca group (Suciu-Foca, N., Int Immunopharmacol, 5:7-11 (2005)). Although the ligand for ILT3 is unknown, ILT4 is known to bind to the third domain of HLA class I molecules (HLA-A, HLA-B, HLA-C, and HLA-G), competing with CD8 for MHC class I binding (Shiroishi, M., Proc Natl Acad Sci USA, 100:8856-8861 (2003)). The preferential ligand for several inhibitory ILT receptors is HLA-G. HLA-G plays a potential role in maternal-fetal tolerance and in the mechanisms of escape of tumor cells from immune recognition and destruction (Hunt, J. S., et al., Faseb J, 19:681-693 (2005)). It is most likely that regulation of DC function by HLA-G-ILT interactions is an important pathway in the biology of DC. It has been determined that human monocyte-derived DC that highly express ILT2 and ILT4 receptors, when treated with HLA-G and stimulated with allogeneic T cells, still maintain a stable tolerogenic-like phenotype (CD80low, CD86low, HLA-DRlow) with the potential to induce T cell anergy (Ristich, V., et al., Eur J Immunol, 35:1133-1142 (2005)). Moreover, the HLA-G interaction with DC that highly express ILT2 and ILT4 receptors resulted in down-regulation of several genes involved in the MHC class II presentation pathway. A lysosomal thiol reductase, IFN-gamma inducible lysosomal thiol reductase (GILT), abundantly expressed by professional APC, was greatly reduced in HLA-G-modified DC. The repertoire of primed CD4+ T cells can be influenced by DC expression of GILT, as in vivo T cell responses to select antigens were reduced in animals lacking GILT after targeted gene disruption (Marie, M., et al., Science, 294:1361-1365 (2001)). The HLA-G/ILT interaction on DC interferes with the assembly and transport of MHC class II molecules to the cell surface, which might result in less efficient presentation or expression of structurally abnormal MHC class II molecules. It was determined that HLA-G markedly decreased the transcription of invariant chain (CD74), HLA-DMA, and HLA-DMB genes on human monocyte-derived DC highly expressing ILT inhibitory receptors (Ristich, V., et al; Eur J Immunol 35:1133-1142 (2005)).

Another receptor of HLA-G is KIR2DL4 because KIR2DL4 binds to cells expressing HLA-G (US2003232051; Cantoni, C. et al. Eur J Immunol 28 (1998) 1980; Rajagopalan, S. and E. O. Long. [published erratum appears in J Exp Med 191 (2000) 2027] J Exp Med 189 (1999) 1093; Ponte, M. et al. PNAS USA 96 (1999) 5674). KIR2DL4 (also referred to as 2DL4) is a KIR family member (also designated CD158d) that shares structural features with both activating and inhibitory receptors (Selvakumar, A. et al. Tissue Antigens 48 (1996) 285). 2DL4 has a cytoplasmic ITIM, suggesting inhibitory function, and a positively charged amino acid in the transmembrane region, a feature typical of activating KIR. Unlike other clonally distributed KIRs, 2DL4 is transcribed by all NK cells (Valiante, N. M. et al. Immunity 7 (1997) 739; Cantoni, C. et al. Eur J Immunol 28 (1998) 1980; Rajagopalan, S. and E. O. Long. [published erratum appears in J Exp Med 191 (2000) 2027] J Exp Med 189 (1999) 1093).

HLA-G has also been shown to interact with CD8 (Sanders et al, J. Exp. Med., 174 (1991), 737-740) on cytotoxic T cells and induce CD95 mediated apoptosis in activated CD8 positive cytotoxic T cells (Fournel et al, J. Immun., 164 (2000), 6100-6104). This mechanism of elimination of cytotoxic T cells has been reported to one of the mechanisms of immune escape and induction of tolerance in pregnancy, inflammatory diseases and cancer (Amodio G. et al, Tissue Antigens, 84 (2014), 255-263).

The term “inhibits ILT2 binding to HLA-G on JEG-3 cells (ATCC HTB36)” refers to the inhibition of binding interaction of (recombinant) ILT2 e.g in an assay as described in Example 5 of WO2022/129120.

As used herein, the terms “first”, “second” or “third” with respect to Fab molecules etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen-binding sites in an antibody. As such, the term “monovalent binding to an antigen” denotes the presence of one (and not more than one) antigen-binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably and refer to an antibody having a structure substantially similar to a native antibody structure.

The term “T cell bispecific antibody (TCB)” or “T cell activating bispecific antibody” as used herein refers to a particular type of multispecific antibody, which is a bispecific antibody designed to simultaneously bind to a surface antigen on a target cell, e.g., a tumor cell, and to an activating, invariant component of the T cell receptor (TCR) complex, such as CD3, for retargeting of T cells to kill target cells. The terms “anti-HLA-G/anti-CD3 antibody”, “anti-HLA-G/anti-CD3 bispecific antibody”, “HLA-G TCB” and “anti-HLA-G TCB” are used interchangeably herein to describe a multispecific antibody, particularly a bispecific antibody, wherein one of the binding specificities is for HLA-G and the other is for CD3. Examples of bispecific antibody formats that may be useful for generating anti-HLA-G/anti-CD3 antibodies include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5 (8) (2016) e1203498.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′) 2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, M A; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen-binding specificity, for example “complementarity determining regions” (“CDRs”). Generally, antibodies comprise six CDRs; three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1 (L1)-FR2-H2 (L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

The term “crossover” Fab molecule (also termed “Crossfab”) as used herein refers a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen-binding moieties) are not the same. In some aspects, the modification promoting association comprises an amino acid mutation in the Fc domain, specifically an amino acid substitution. In a particular aspect, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package is publicly available from http://fasta.bioch.virginia.edu/fasta_www2/fasta_down.shtml and was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36. Alternatively, a public server accessible at http://fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Reduced binding”, for example, reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ (SEQ ID NO: 62), (SG₄)ₙ (SEQ ID NO: 63) or G₄(SG₄)ₙ (SEQ ID NO: 64) peptide linkers, wherein “n” is generally a number between 1 and 10, typically between 2 and 4, in particular 2. Peptide linkers of particular interest are GG, GGS, GSGS (SEQ ID NO: 61), (G₄S) (SEQ ID NO: 56), (G₄S)₂ (SEQ ID NO: 57), (G₄S)₃ (SEQ ID NO: 58), (G₄S)₄ (SEQ ID NO: 59) and GGGGSGGGGG (SEQ ID NO: 60), more particularly (G₄S)₂ (SEQ ID NO:57).

In one aspect, the anti-HLA-G/anti-CD3 bispecific antibody comprises a first antigen-binding moiety that specifically binds to CD3, and a second, and optionally a third, antigen-binding moiety that specifically binds to HLA-G, particularly to HLA-G expressed on JEG3 cells (ATCC No. HTB36), and inhibits ILT2 binding to HLA-G expressed on JEG-3 cells (ATCC No. HTB36).

In another aspect, the first antigen-binding moiety comprises a heavy chain variable region comprising the heavy chain CDR (CDR-H) 1 of SEQ ID NO: 1, the CDR-H2 of SEQ ID NO: 2, and the CDR-H3 of SEQ ID NO: 3; and a light chain variable region comprising the light chain CDR (CDR-L) 1 of SEQ ID NO: 4, the CDR-L2 of SEQ ID NO: 5 and the CDR-L3 of SEQ ID NO: 6.

In a further aspect, the second and (where present) third antigen-binding moiety comprises a heavy chain variable region comprising the heavy chain CDR (CDR-H) 1 of SEQ ID NO: 9, the CDR-H2 of SEQ ID NO: 10, and the CDR-H3 of SEQ ID NO: 11; and a light chain variable region comprising the light chain CDR (CDR-L) 1 of SEQ ID NO: 12, the CDR-L2 of SEQ ID NO: 13 and the CDR-L3 of SEQ ID NO: 14.

In a particular aspect, the anti-HLA-G/anti-CD3 bispecific antibody comprises
(i) a first antigen-binding moiety that specifically binds to CD3 and comprises a heavy chain variable region comprising the heavy chain CDR (CDR-H) 1 of SEQ ID NO: 1, the CDR-H2 of SEQ ID NO: 2, and the CDR-H3 of SEQ ID NO: 3; and a light chain variable region comprising the light chain CDR (CDR-L) 1 of SEQ ID NO: 4, the CDR-L2 of SEQ ID NO: 5 and the CDR-L3 of SEQ ID NO: 6; and
(ii) a second, and optionally a third, antigen-binding moiety that specifically binds to HLA-G and comprises a heavy chain variable region comprising the heavy chain CDR (CDR-H) 1 of SEQ ID NO: 9, the CDR-H2 of SEQ ID NO: 10, and the CDR-H3 of SEQ ID NO: 11; and a light chain variable region comprising the light chain CDR (CDR-L) 1 of SEQ ID NO: 12, the CDR-L2 of SEQ ID NO: 13 and the CDR-L3 of SEQ ID NO: 14.

In one aspect, the first antigen-binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen-binding moiety comprises the heavy chain variable region sequence of SEQ ID NO: 7 and the light chain variable region sequence of SEQ ID NO: 8.

In one aspect, the second and (where present) third antigen-binding moiety comprises a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one aspect, the second and (where present) third antigen-binding moiety comprises the heavy chain variable region sequence of SEQ ID NO: 15 and the light chain variable region sequence of SEQ ID NO: 16.

In some aspects, the first, the second and/or (where present) the third antigen-binding moiety is a Fab molecule. In some aspects, the first antigen-binding moiety is a crossover Fab molecule wherein either the variable or the constant regions of the Fab light chain and the Fab heavy chain are exchanged. In such aspects, the second and (where present) third antigen-binding moiety preferably is a conventional Fab molecule.

In some aspects wherein the first, the second and (where present) the third antigen-binding moiety of the bispecific antibody are all Fab molecules, and in one of the antigen-binding moieties (particularly the first antigen-binding moiety) the variable domains VL and VH of the Fab light chain and the Fab heavy chain are replaced by each other,
i) the amino acid at position 124 in the constant domain CL of the first antigen-binding moiety is substituted by a positively charged amino acid (numbering according to Kabat), and the amino acid at position 147 or the amino acid at position 213 in the constant domain CH1 of the first antigen-binding moiety is substituted by a negatively charged amino acid (numbering according to Kabat EU index); or
ii) the amino acid at position 124 in the constant domain CL of the second and/or (where present) third antigen-binding moiety is substituted by a positively charged amino acid (numbering according to Kabat), and the amino acid at position 147 or the amino acid at position 213 in the constant domain CH1 of the second and/or (where present) third antigen-binding moiety is substituted by a negatively charged amino acid (numbering according to Kabat EU index).

The bispecific antibody does not comprise both modifications mentioned under i) and ii). The constant domains CL and CH1 of the antigen-binding moiety having the VH/VL exchange are not replaced by each other (i.e. remain unexchanged).

In a more specific aspect,
i) the amino acid at position 124 in the constant domain CL of the first antigen-binding moiety is substituted independently by lysine (K), arginine (R) or histidine (H) (numbering according to Kabat), and the amino acid at position 147 or the amino acid at position 213 in the constant domain CH1 of the first antigen-binding moiety is substituted independently by glutamic acid (E), or aspartic acid (D) (numbering according to Kabat EU index); or
ii) the amino acid at position 124 in the constant domain CL of the second and/or (where present) third antigen-binding moiety is substituted independently by lysine (K), arginine (R) or histidine (H) (numbering according to Kabat), and the amino acid at position 147 or the amino acid at position 213 in the constant domain CH1 of the second antigen-binding moiety is substituted independently by glutamic acid (E), or aspartic acid (D) (numbering according to Kabat EU index).

In one such aspect, the amino acid at position 124 in the constant domain CL of the first antigen-binding moiety is substituted independently by lysine (K), arginine (R) or histidine (H) (numbering according to Kabat), and the amino acid at position 147 or the amino acid at position 213 in the constant domain CH1 of the first third antigen-binding moiety is substituted independently by glutamic acid (E), or aspartic acid (D) (numbering according to Kabat EU index).

In a further aspect, in the constant domain CL of the second and/or (where present) third antigen-binding moiety the amino acid at position 124 is substituted independently by lysine (K), arginine (R) or histidine (H) (numbering according to Kabat), and in the constant domain CH1 of the second and/or (where present) third antigen-binding moiety the amino acid at position 147 is substituted independently by glutamic acid (E), or aspartic acid (D) (numbering according to Kabat EU index).

In preferred aspects, in the constant domain CL of the second and/or (where present) third antigen-binding moiety the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second and/or (where present) third antigen-binding moiety the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the constant domain CL of the second and/or (where present) third antigen-binding moiety the amino acid at position 124 is substituted by lysine (K) (numbering according to Kabat) and the amino acid at position 123 is substituted by lysine (K) (numbering according to Kabat), and in the constant domain CH1 of the second and/or (where present) third antigen-binding moiety the amino acid at position 147 is substituted by glutamic acid (E) (numbering according to Kabat EU index) and the amino acid at position 213 is substituted by glutamic acid (E) (numbering according to Kabat EU index).

In one aspect, in the constant domain CL of the second and/or (where present) third antigen-binding moiety the amino acid at position 124 is substituted by lysine (K) (numbering according to Kabat) and the amino acid at position 123 is substituted by arginine (R) (numbering according to Kabat), and in the constant domain CH1 of the second and/or (where present) third antigen-binding moiety the amino acid at position 147 is substituted by glutamic acid (E) (numbering according to Kabat EU index) and the amino acid at position 213 is substituted by glutamic acid (E) (numbering according to Kabat EU index).

In particular aspects, if amino acid substitutions according to the above aspects are made in the constant domain CL and the constant domain CH1 of the second and/or (where present) third antigen-binding moiety, the constant domain CL of the second and/or (where present) the third antigen-binding moiety is of the kappa isotype.

In some aspects, the first and the second antigen-binding moiety are fused to each other, optionally via a peptide linker.

In some aspects, the first and the second antigen-binding moiety are each a Fab molecule and either (i) the second antigen-binding moiety is fused at the C-terminus of the Fab heavy chain to the N-terminus of the Fab heavy chain of the first antigen-binding moiety, or (ii) the first antigen-binding moiety is fused at the C-terminus of the Fab heavy chain to the N-terminus of the Fab heavy chain of the second antigen-binding moiety.

In some aspects, the anti-HLA-G/anti-CD3 bispecific antibody provides monovalent binding to CD3.

In particular aspects, the anti-HLA-G/anti-CD3 bispecific antibody comprises a single antigen-binding moiety that specifically binds to CD3, and two antigen-binding moieties that specifically bind to HLA-G. Thus, in some aspects, the anti-HLA-G/anti-CD3 bispecific antibody comprises a third antigen-binding moiety, particularly a Fab molecule, more particularly a conventional Fab molecule, that specifically binds to HLA-G. The third antigen-binding moiety may incorporate, singly or in combination, all of the features described hereinabove in relation to the second antigen-binding moiety (e.g. the CDR sequences, variable region sequences, and/or amino acid substitutions in the constant regions). In some aspects, the third antigen moiety is identical to the second antigen-binding moiety (e.g. is also a conventional Fab molecule and comprises the same amino acid sequences).

In particular aspects, the anti-HLA-G/anti-CD3 bispecific antibody further comprises an Fc domain composed of a first and a second subunit. In one aspect, the Fc domain is an IgG Fc domain. In a particular aspect, the Fc domain is an IgG₁ Fc domain. In another aspect, the Fc domain is an IgG₄ Fc domain. In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular aspect, the Fc domain is a human Fc domain. In a particularly preferred aspect, the Fc domain is a human IgG₁ Fc domain. An exemplary sequence of a human IgG₁ Fc region is given in SEQ ID NO: 43.

In some aspects wherein the first and the second antigen-binding moiety are each a Fab molecule, the first antigen-binding moiety is fused at the C-terminus of the Fab heavy chain to the N-terminus of the first subunit of the Fc domain, and the second antigen-binding moiety is fused at the C-terminus of the Fab heavy chain to the N-terminus of the second subunit of the Fc domain.

In some aspects wherein the first, the second and, where present, the third antigen-binding moiety are each a Fab molecule, (a) either (i) the second antigen-binding moiety is fused at the C-terminus of the Fab heavy chain to the N-terminus of the Fab heavy chain of the first antigen-binding moiety and the first antigen-binding moiety is fused at the C-terminus of the Fab heavy chain to the N-terminus of the first subunit of the Fc domain, or (ii) the first antigen-binding moiety is fused at the C-terminus of the Fab heavy chain to the N-terminus of the Fab heavy chain of the second antigen-binding moiety and the second antigen-binding moiety is fused at the C-terminus of the Fab heavy chain to the N-terminus of the first subunit of the Fc domain; and (b) the third antigen-binding moiety, where present, is fused at the C-terminus of the Fab heavy chain to the N-terminus of the second subunit of the Fc domain.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in one aspect said modification is in the CH3 domain of the Fc domain.

In a specific aspect, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g. by site-specific mutagenesis, or by peptide synthesis.

In a specific such aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function.

In a particular aspect, the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular aspect, the effector function is ADCC.

Typically, the same one or more amino acid substitution is present in each of the two subunits of the Fc domain. In one aspect, the one or more amino acid substitution reduces the binding affinity of the Fc domain to an Fc receptor. In one aspect, the one or more amino acid substitution reduces the binding affinity of the Fc domain to an Fc receptor by at least 2-fold, at least 5-fold, or at least 10-fold.

In one aspect, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In one such aspect, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such aspect, the Fc domain is an IgG₁ Fc domain, particularly a human IgG₁ Fc domain.

In preferred aspects, the anti-HLA-G/anti-CD3 bispecific antibody comprises
(i) a first antigen-binding moiety that specifically binds to CD3, comprising a heavy chain variable region comprising the heavy chain CDR (CDR-H) 1 of SEQ ID NO: 1, the CDR-H2 of SEQ ID NO: 2, and the CDR-H3 of SEQ ID NO: 3; and a light chain variable region comprising the light chain CDR (CDR-L) 1 of SEQ ID NO: 4, the CDR-L2 of SEQ ID NO: 5 and the CDR-L3 of SEQ ID NO: 6, wherein the first antigen-binding moiety is a crossover Fab molecule wherein either the variable or the constant regions, particularly the variable regions, of the Fab light chain and the Fab heavy chain are exchanged;
(ii) a second and a third antigen-binding moiety that specifically bind to HLA-G, comprising a heavy chain variable region comprising the heavy chain CDR (CDR-H) 1 of SEQ ID NO: 9, the CDR-H2 of SEQ ID NO: 10, and the CDR-H3 of SEQ ID NO: 11; and a light chain variable region comprising the light chain CDR (CDR-L) 1 of SEQ ID NO: 12, the CDR-L2 of SEQ ID NO: 13 and the CDR-L3 of SEQ ID NO: 14, wherein the second and third antigen-binding moiety are each a Fab molecule, particularly a conventional Fab molecule;
(iii) an Fc domain composed of a first and a second subunit,
wherein the second antigen-binding moiety is fused at the C-terminus of the Fab heavy chain to the N-terminus of the Fab heavy chain of the first antigen-binding moiety, and the first antigen-binding moiety is fused at the C-terminus of the Fab heavy chain to the N-terminus of the first subunit of the Fc domain, and wherein the third antigen-binding moiety is fused at the C-terminus of the Fab heavy chain to the N-terminus of the second subunit of the Fc domain.

In one aspect, the first antigen-binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen-binding moiety comprises the heavy chain variable region sequence of SEQ ID NO: 7 and the light chain variable region sequence of SEQ ID NO: 8.

In one aspect, the second and third antigen-binding moiety comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one aspect, the second and third antigen-binding moieties comprise the heavy chain variable region of SEQ ID NO: 15 and the light chain variable region of SEQ ID NO: 16.

The Fc domain according to the above aspects may incorporate, singly or in combination, all of the features described hereinabove in relation to Fc domains.

In one aspect, the antigen-binding moieties and the Fc region are fused to each other by peptide linkers, particularly by peptide linkers as in SEQ ID NO: 56-64.

In one aspect, in the constant domain CL of the second and the third Fab molecule under (ii) the amino acid at position 124 is substituted by lysine (K) (numbering according to Kabat) and the amino acid at position 123 is substituted by lysine (K) or arginine (R), particularly by arginine (R) (numbering according to Kabat), and in the constant domain CH1 of the second and the third Fab molecule under (ii) the amino acid at position 147 is substituted by glutamic acid (E) (numbering according to Kabat EU index) and the amino acid at position 213 is substituted by glutamic acid (E) (numbering according to Kabat EU index).

In one aspect, the anti-HLA-G/anti-CD3 bispecific antibody comprises a polypeptide (particularly two polypeptides) comprising a sequence that is at least 80%, 85%, 90%, 95%, 96%, 97%, 98%, or 99% identical to the sequence of SEQ ID NO: 46, a polypeptide comprising a sequence that is at least 80%, 85%, 90%, 95%, 96%, 97%, 98%, or 99% identical to the sequence of SEQ ID NO: 47, a polypeptide comprising a sequence that is at least 80%, 85%, 90%, 95%, 96%, 97%, 98%, or 99% identical to the sequence of SEQ ID NO: 48, and a polypeptide comprising a sequence that is at least 80%, 85%, 90%, 95%, 96%, 97%, 98%, or 99% identical to the sequence of SEQ ID NO: 49.

In one aspect, the anti-HLA-G/anti-CD3 bispecific antibody comprises a polypeptide (particularly two polypeptides) comprising the sequence of SEQ ID NO: 46, a polypeptide comprising the sequence of SEQ ID NO: 47, a polypeptide comprising the sequence of SEQ ID NO: 48, and a polypeptide comprising the sequence of SEQ ID NO: 49.

The anti-HLA-G/anti-CD3 bispecific antibody herein is used in combination with a 4-1BB (CD137) agonist.

The term “4-1BB” or “CD137”, as used herein, refers to any native 4-1BB from any vertebrate source, including mammals such as primates (e.g. humans) and rodents (e.g., mice and rats), unless otherwise indicated. The term encompasses “full-length,” unprocessed 4-1BB as well as any form of 4-1BB that results from processing in the cell. The term also encompasses naturally occurring variants of 4-1BB, e.g., splice variants or allelic variants. The amino acid sequence of a human 4-1BB is shown in UniProt accession no. Q07011 (entry version 185).

“4-1BBL” or “4-1BB ligand” or “CD137L” is a costimulatory TNF ligand family member, which is able to costimulate proliferation and cytokine production of T cells. Costimulatory TNF family ligands can costimulate TCR signals upon interaction with their corresponding TNF receptors and the interaction with their receptors leads to recruitment of TNFR-associated factors (TRAF), which initiate signalling cascades that result in T cell activation. 4-1BBL is a type II transmembrane protein. Complete or full-length 4-1BBL having the amino acid sequence shown in UniProt accession no. P41273 (entry version 153) has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO: 55) form the ectodomain of 4-1BBL, but even fragments thereof are able to form the trimers.

An “ectodomain” is the domain of a membrane protein that extends into the extracellular space (i.e. the space outside the cell), also referred to as “extracellular domain”. The ectodomain of 4-1BBL as defined herein refers to the part of the 4-1BBL protein, particularly the human 4-1BBL protein (UniProt accession no. P41273 (entry version 153)) that extends into the extracellular space (the extracellular domain), but also includes shorter parts or fragments thereof that are responsible for the trimerization and for the binding to the corresponding receptor 4-1BB.

The term “ectodomain of 4-1BBL or a fragment thereof” thus refers to the extracellular domain domain of 4-1BBL, or to parts thereof that are able to bind to 4-1BB and are capable of trimerization. In specific aspects of the invention, the term “ectodomain of 4-1BBL or a fragment thereof” refers to a polypeptide having an amino acid sequence selected from SEQ ID NO: 25 (amino acids 71-254 of human 4-1BBL), SEQ ID NO: 26 (amino acids 85-254 of human 4-1BBL), SEQ ID NO: 27 (amino acids 80-254 of human 4-1BBL), SEQ ID NO: 28 (amino acids 52-254 of human 4-1BBL), SEQ ID NO: 29 (amino acids 71-248 of human 4-1BBL), SEQ ID NO: 30 (amino acids 85-248 of human 4-1BBL), SEQ ID NO: 31 (amino acids 80-248 of human 4-1BBL) and SEQ ID NO: 32 (amino acids 52-248 of human 4-1BBL).

As used herein, the term “antigen-binding molecule” refers in its broadest sense to a molecule that specifically binds an antigenic determinant. Examples of antigen-binding molecules are antibodies, antibody fragments and scaffold antigen-binding proteins.

“Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. The amino acid sequence of human FAP is shown in UniProt (www.uniprot.org) accession no. Q12884 (entry version 197). The extracellular domain (ECD) of human FAP extends from amino acid position 26 to 760. An antigen-binding moiety that binds to FAP as used herein preferably binds to the extracellular domain of FAP. Exemplary anti-FAP binding molecules are described e.g. in PCT publication no. WO 2012/020006.

Particularly useful 4-1BB (CD137) agonists for use in the present invention are described e.g. in PCT publication no. WO 2016/075278 or in PCT publication no. WO 2016/156291 (incorporated herein by reference in their entirety).

In one aspect, the 4-1BB (CD137) agonist comprises 4-1BBL (in particular human 4-1BBL) or a fragment thereof, particularly an ectodomain of 4-1BBL or fragment thereof. In one aspect, the 4-1BB (CD137) agonist comprises three ectodomains of 4-1BBL or fragments thereof (i.e. a first, a second and a third ectodomain of 4-1BBL or fragment thereof). In one aspect, the ectodomain of 4-1BBL or fragment thereof comprises an amino acid sequence that is at least about 95%, 96%, 97%, 98%, 99% or 100% identical to an amino acid sequence selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, and SEQ ID NO: 32. In one aspect, the ectodomain of 4-1BBL of fragment thereof comprises an amino acid sequence that is at least about 95%, 96%, 97%, 98%, 99% or 100% identical to the amino acid sequence of SEQ ID NO: 29. In one aspect, the ectodomain of 4-1BBL or fragment thereof comprises the amino acid sequence of SEQ ID NO: 29. In one aspect, the ectodomain of 4-1BBL or fragment thereof consists of the amino acid sequence of SEQ ID NO: 29.

In a particular aspect, the 4-1BB (CD137) agonist is a molecule comprising three ectodomains of 4-1BBL or fragments thereof, wherein the ectodomains of 4-1BBL or fragments thereof comprise (or consist of) the amino acid sequence of SEQ ID NO: 29.

In one aspect, the 4-1BB (CD137) agonist comprises three ectodomains of 4-1BBL or fragments thereof (i.e. a first, a second and a third ectodomain of 4-1BBL or fragment thereof), wherein the first and the second ectodomain of 4-1BBL or fragment thereof are fused to each other, optionally via a peptide linker (i.e. the first and the second ectodomain of 4-1BBL or fragment thereof are on the same polypeptide), and the third ectodomain of 4-1BBL or fragment thereof is not fused to the first or the second ectodomain of 4-1BBL or fragment thereof (i.e. the third ectodomain of 4-1BBL or fragment thereof is on a separate polypeptide from the first and the second ectodomain of 4-1BBL or fragment thereof).

In one aspect, the 4-1BB (CD137) agonist is a molecule comprising a first polypeptide comprising a first and a second ectodomain of 4-1BBL or fragment thereof, and a second polypeptide comprising a third ectodomain of 4-1BBL or fragment thereof. In one aspect, the first and the second ectodomain of 4-1BBL or fragment thereof are fused via a peptide linker, particularly a (G4S) 2 peptide linker (SEQ ID NO: 57). In one aspect, the first and the second polypeptide are linked by a disulfide bond. In one aspect, the first polypeptide comprises an amino acid sequence that is at least about 95%, 96%, 97%, 98%, 99% or 100% identical to the amino acid sequence of SEQ ID NO: 37. In one aspect, the first polypeptide comprises the amino acid sequence of SEQ ID NO: 37. In one aspect, the second polypeptide comprises an amino acid sequence that is at least about 95%, 96%, 97%, 98%, 99% or 100% identical to the amino acid sequence of SEQ ID NO: 29. In one aspect, the second polypeptide comprises the amino acid sequence of SEQ ID NO: 29.

In a further aspect, the 4-1BB (CD137) agonist comprises an antigen-binding moiety. In a particular aspect, the 4-1BB (CD137) agonist comprises an antigen-binding moiety that specifically binds to a tumor-associated antigen, particularly a tumor stromal antigen (i.e. an antigen associated with the tumor stroma), more particularly a tumor fibroblast antigen (i.e. an antigen expressed on cancer-associated fibroblasts).

In a preferred aspect, the antigen-binding moiety specifically binds to Fibroblast Activation Protein (FAP), particularly human FAP.

In one aspect, the antigen-binding moiety is a Fab molecule, particularly a conventional Fab molecule.

Thus, in a particular aspect, the 4-1BB (CD137) agonist is an antigen-binding molecule comprising three ectodomains of 4-1BBL or fragments thereof, and at least one antigen-binding moiety that specifically binds to a tumor-associated antigen, in particular an antigen-binding moiety that specifically binds to FAP.

In one aspect, the antigen-binding moiety that specifically binds to FAP comprises a heavy chain variable region (VH) comprising the heavy chain CDR (CDR-H) 1 of SEQ ID NO: 17, the CDR-H2 of SEQ ID NO: 18, and the CDR-H3 of SEQ ID NO: 19; and a light chain variable region comprising the light chain CDR (CDR-L) 1 of SEQ ID NO: 20, the CDR-L2 of SEQ ID NO: 21 and the CDR-L3 of SEQ ID NO: 22.

In one aspect, the antigen-binding moiety that specifically binds to FAP comprises a heavy chain variable region sequence that is at least about 95%, 96%, 97%, 98%, 99% or 100% identical to the amino acid sequence of SEQ ID NO: 23 and a light chain variable region sequence that is at least about 95%, 96%, 97%, 98%, 99% or 100% identical to the amino acid sequence of SEQ ID NO: 24.

In one aspect, the antigen-binding moiety that specifically binds to FAP comprises the heavy chain variable region sequence of SEQ ID NO: 23 and the light chain variable region sequence of SEQ ID NO: 24.

In a further aspect, the 4-1BB (CD137) agonist comprises an Fc domain composed of a first and a second subunit.

The Fc domain comprised in the 4-1BB (CD137) agonist may incorporate, singly or in combination, all of the features described hereinabove in relation to Fc domains comprised in the anti-HLA-G/anti-CD3 bispecific antibody.

In particular, in one aspect, the Fc domain comprised in the 4-1BB (CD137) agonist is an IgG Fc domain. In a particular aspect, the Fc domain is an IgG₁ Fc domain. In a further particular aspect, the Fc domain is a human Fc domain. In a particularly preferred aspect, the Fc domain is a human IgG₁ Fc domain.

In a particular aspect, the Fc domain comprises a modification promoting the association of the first and the second subunit of the Fc domain (such as the “knob-into-hole” modification), as described hereinabove in relation to the anti-HLA-G/anti-CD3 bispecific antibody.

In a further particular aspect, the Fc domain comprises one or more amino acid substitution that reduces binding to an Fc receptor and/or effector function (such as the “P329G LALA”, “PGLALA” or “LALAPG” amino acid substitutions), as described hereinabove in relation to the anti-HLA-G/anti-CD3 bispecific antibody.

In a particularly preferred aspect, the Fc domain comprised in the 4-1BB (CD137) agonist is a human IgG₁ Fc domain, wherein each subunit of the Fc domain comprises the amino acid substitutions L234A, L235A and P329G (Kabat EU index numbering).

In one aspect, the 4-1BB (CD137) agonist is an antigen-binding molecule comprising
(i) three ectodomains of 4-1BBL or fragments thereof;
(ii) an antigen-binding moiety that specifically binds to FAP, particularly wherein the antigen-binding moiety is a Fab molecule; and
(iii) an Fc domain composed of a first and a second subunit, particularly wherein the Fc domain comprises a modification promoting the association of the first and the second subunit of the Fc domain and/or one or more amino acid substitution that reduces binding to an Fc receptor and/or effector function.

In a particular aspect, the 4-1BB (CD137) agonist is an antigen-binding molecule comprising
(i) a first, a second and a third ectodomain of 4-1BBL or fragment thereof;
(ii) an antigen-binding moiety that specifically binds to FAP, wherein the antigen-binding moiety is a Fab molecule;
(iii) an Fc domain composed of a first and a second subunit, particularly wherein the Fc domain comprises a modification promoting the association of the first and the second subunit of the Fc domain and/or one or more amino acid substitution that reduces binding to an Fc receptor and/or effector function;
(iv) a CL domain and a CH1 domain;
wherein the antigen-binding molecule is composed of
(a) a first polypeptide, comprising the
   (a1) first ectodomain of 4-1BBL or fragment thereof, fused at its C-terminus to the N-terminus of the second ectodomain of 4-1BBL or fragment thereof,
   (a2) the second ectodomain of 4-1BBL or fragment thereof, fused at its C-terminus to the N-terminus of the CL domain,
   (a3) the CL domain, fused at its C-terminus to the N-terminus of one of the subunits (e.g. the first subunit) of the Fc domain, and
   (a4) one of the subunits (e.g. the first subunit) of the Fc domain;
(b) a second polypeptide, comprising
   (b1) the third ectodomain of 4-1BBL or fragment thereof, fused at its C-terminus to the N-terminus of the CH1 domain, and
   (b2) the CH1 domain;
(c) a third polypeptide, comprising
   (c1) the heavy chain of the Fab molecule, fused at its C-terminus to the N-terminus of the other one of the subunits (e.g. the second subunit) of the Fc domain, and
   (c2) the other one of the subunits (e.g. the second subunit) of the Fc domain; and
(d) a fourth polypeptide, comprising the light chain of the Fab molecule.

Fusion between the various domains of the antigen-binding molecule is preferably via peptide linkers, which may also comprise or consist of (part of) an immunoglobulin hinge region. In particular, fusion of the first and the second ectodomain of 4-1BBL or fragment thereof is via a peptide linker, particularly a (G4S) 2 linker (SEQ ID NO: 57).

In one aspect, the first and the second polypeptide are linked to each other by a disulfide bond, particularly a disulfide bond between the CL domain and the CH1 domain.

In one aspect, in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the CL domain of the first polypeptide the amino acid at position 124 is substituted by lysine (K) (numbering according to Kabat) and the amino acid at position 123 is substituted by lysine (K) (numbering according to Kabat), and in the CH1 domain of the second polypeptide the amino acid at position 147 is substituted by glutamic acid (E) (numbering according to Kabat EU index) and the amino acid at position 213 is substituted by glutamic acid (E) (numbering according to Kabat EU index).

In a particular aspect, in the CL domain of the first polypeptide the amino acid at position 124 is substituted by lysine (K) (numbering according to Kabat) and the amino acid at position 123 is substituted by arginine (R) (numbering according to Kabat), and in the CH1 domain of the second polypeptide the amino acid at position 147 is substituted by glutamic acid (E) (numbering according to Kabat EU index) and the amino acid at position 213 is substituted by glutamic acid (E) (numbering according to Kabat EU index).

In a particular aspect, if amino acid substitutions according to the above aspects are made in the CL and CH1 domain of the first and second polypeptide, the CL domain of the first polypeptide is of kappa isotype.

In one aspect, the CL domain is a human CL domain, particularly a human CL domain of kappa isotype. In a further aspect, the CH1 domain is a human CH1 domain, particularly a human CH1 domain of γ isotype, most particularly a human CH1 domain of γ1 isotype.

In one aspect, the first polypeptide comprises an amino acid sequence that is at least about 95%, 96%, 97%, 98%, 99% or 100% identical to the amino acid sequence of SEQ ID NO: 51.

In one aspect, the second polypeptide comprises an amino acid sequence that is at least about 95%, 96%, 97%, 98%, 99% or 100% identical to the amino acid sequence of SEQ ID NO: 52.

In one aspect, the third polypeptide comprises an amino acid sequence that is at least about 95%, 96%, 97%, 98%, 99% or 100% identical to the amino acid sequence of SEQ ID NO: 53.

In one aspect, the fourth polypeptide comprises an amino acid sequence that is at least about 95%, 96%, 97%, 98%, 99% or 100% identical to the amino acid sequence of SEQ ID NO: 54.

In one aspect, the 4-1BB (CD137) agonist is an antigen-binding molecule comprising a first polypeptide comprising an amino acid sequence that is at least about 95%, 96%, 97%, 98%, 99% or 100% identical to the amino acid sequence of SEQ ID NO: 51, a second polypeptide comprising an amino acid sequence that is at least about 95%, 96%, 97%, 98%, 99% or 100% identical to the amino acid sequence of SEQ ID NO: 52, a third polypeptide comprising an amino acid sequence that is at least about 95%, 96%, 97%, 98%, 99% or 100% identical to the amino acid sequence of SEQ ID NO: 53, and a fourth polypeptide comprising an amino acid sequence that is at least about 95%, 96%, 97%, 98%, 99% or 100% identical to the amino acid sequence of SEQ ID NO: 54.

In one aspect, the 4-1BB (CD137) agonist comprises a first polypeptide comprising the amino acid sequence of SEQ ID NO: 51, a second polypeptide comprising the amino acid sequence of SEQ ID NO: 52, a third polypeptide comprising the amino acid sequence of SEQ ID NO: 53, and a fourth polypeptide comprising the amino acid sequence of SEQ ID NO: 54.

In alternative aspects, the 4-1BB agonist may be an anti-4-1BB antibody, particularly an anti-FAP/anti-4-1BB bispecific antibody.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In some aspects of the anti-HLA-G/anti-CD3 bispecific antibodies, 4-1BB (CD137) agonists, methods, uses and kits of the invention, the cancer is a solid tumor cancer. By a “solid tumor cancer” is meant a malignancy that forms a discrete tumor mass (including also tumor metastasis) located at specific location in the patient's body, such as sarcomas or carcinomas (as opposed to e.g. blood cancers such as leukemia, which generally do not form solid tumors). Non-limiting examples of solid tumor cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, bone cancer, liver cancer and kidney cancer (renal cancer). Other solid tumor cancers that are contemplated in the context of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, muscles, spleen, thoracic region, and urogenital system. Also included are pre-cancerous conditions or lesions and cancer metastases.

In one aspect, the cancer is a cancer selected from the group consisting of renal cell carcinoma, colorectal carcinoma, non-small cell lung cancer and pancreatic duct adenocarcinoma (PDAC), head and neck cancer, bladder cancer, esophageal cancer, skin cancer, soft tissue cancer, gastric cancer, cervical cancer and ovarian cancer. In one aspect, the cancer is a cancer selected from the group consisting of renal cell carcinoma, colorectal carcinoma, non-small cell lung cancer and pancreatic duct adenocarcinoma (PDAC).

In some aspects, the cancer is a HLA-G-positive cancer. By “HLA-G-positive cancer” or “HLA-G-expressing cancer” is meant a cancer characterized by expression or overexpression of HLA-G in cancer cells. The expression of HLA-G may be determined for example by quantitative real-time PCR (measuring HLA-G mRNA levels), immunohistochemistry (IHC) or western blot assays. In one aspect, the cancer expresses HLA-G. In one aspect, the cancer expresses HLA-G in at least 20%, preferably at least 50% or at least 80% of tumor cells as determined by immunohistochemistry (IHC) using an antibody specific for HLA-G. In one embodiment, HLA-G expressing cancers are selected from renal cell carcinoma, colorectal carcinoma, non-small cell lung cancer and pancreatic duct adenocarcinoma (PDAC).

In some aspects, the cancer comprises cells (e.g. fibroblasts) expressing FAP. In some aspects, the cancer expresses FAP, particularly in the tumor stroma.

A “patient”, “subject” or “individual” herein is any single human subject eligible for treatment who is experiencing or has experienced one or more signs, symptoms, or other indicators of cancer. In some aspects, the patient has cancer or has been diagnosed with cancer. The patient may have been previously treated with an anti-HLA-G/anti-CD3 bispecific antibody or another drug, or not so treated. In particular aspects, the patient has not been previously treated with an anti-HLA-G/anti-CD3 bispecific antibody. The patient may have been treated with a therapy comprising one or more drugs other than an anti-HLA-G/anti-CD3 bispecific antibody before the anti-HLA-G/anti-CD3 bispecific antibody therapy is commenced.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The anti-HLA-G/anti-CD3 bispecific antibody and the 4-1BB (CD137) agonist are administered in an effective amount.

An “effective amount” of an agent, e.g. a pharmaceutical composition, refers to an amount effective, at dosages and for periods of time necessary, to achieve the desired therapeutic or prophylactic result.

In some aspects of the anti-HLA-G/anti-CD3 bispecific antibodies, 4-1BB (CD137) agonists, methods, uses or kits described above and herein, the treatment with or administration of the anti-HLA-G/anti-CD3 bispecific antibody and the 4-1BB (CD137) agonist results in an increase of tumor-infiltrating immune cell numbers, particularly of CD45+ cell numbers, at the site of the cancer. In one aspect, the treatment with or administration of the anti-HLA-G/anti-CD3 bispecific antibody and the 4-1BB (CD137) agonist results in an increase of T cell numbers, particularly cytotoxic T cell and/or CD4+ T cell numbers, at the site of the cancer. In one aspect, the treatment with or administration of the anti-HLA-G/anti-CD3 bispecific antibody and the 4-1BB (CD137) agonist results in activation of T cells, particularly cytotoxic T cells, particularly at the site of the cancer. Said activation may comprise proliferation of T cells, differentiation of T cells, cytokine secretion by T cells, cytotoxic effector molecule release from T cells, cytotoxic activity of T cells, and expression of activation markers by T cells. In some aspects of the anti-HLA-G/anti-CD3 bispecific antibodies, 4-1BB (CD137) agonists, methods, uses or kits described above and herein, the treatment with or administration of the anti-HLA-G/anti-CD3 bispecific antibody and the 4-1BB (CD137) agonist results in increased activation of T cells, particularly cytotoxic T cells, particularly at the site of the cancer, as compared to treatment with or administration of the anti-HLA-G/anti-CD3 bispecific antibody alone. In particular aspects, the activation comprises cytotoxic activity (specifically lysis of cancer cells) of T cells, including Granzyme B release and/or cytokine (specifically IL-2, TNF-α, and/or interferon-γ) secretion by T cells. In some aspects of the anti-HLA-G/anti-CD3 bispecific antibodies, 4-1BB (CD137) agonists, methods, uses or kits described above and herein, the treatment with or administration of the anti-HLA-G/anti-CD3 bispecific antibody and the 4-1BB (CD137) agonist results in reduced macrophage infiltration of the tumor, in particular reduced macrophage infiltration of the tumor with macrophages that are CD68+. Said macrophage infiltration may comprise a decrease of macrophage-mediated T cell suppression and/or an increase of T cell activation.

In some aspects of the anti-HLA-G/anti-CD3 bispecific antibodies, 4-1BB (CD137) agonists, methods, uses or kits described above and herein, the treatment with or administration of the anti-HLA-G/anti-CD3 bispecific antibody and the 4-1BB (CD137) agonist results in increased differentiation of naïve T cells towards memory T cells, particularly at the site of the cancer, as compared to treatment with or administration of the anti-HLA-G/anti-CD3 bispecific antibody alone. In one aspect, the differentiation is detected by measurement of CD45RA expression, e.g. using flow cytometry.

In some aspects of the anti-HLA-G/anti-CD3 bispecific antibodies, 4-1BB (CD137) agonists, methods, uses or kits described above and herein, the treatment with or administration of the anti-HLA-G/anti-CD3 bispecific antibody and the 4-1BB (CD137) agonist may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the anti-HLA-G/anti-CD3 bispecific antibody and the 4-1BB (CD137) agonist may improve the response as compared to treatment with or administration of the anti-HLA-G/anti-CD3 bispecific antibody alone (i.e. without the 4-1BB (CD137) agonist).

In some aspects, the treatment or administration of the anti-HLA-G/anti-CD3 bispecific antibody and the 4-1BB (CD137) agonist may increase response rates in a patient population, as compared to a corresponding patient population treated with the anti-HLA-G/anti-CD3 bispecific antibody alone (i.e. without the 4-1BB (CD137) agonist).

The combination therapy of the invention comprises administration of an anti-HLA-G/anti-CD3 bispecific antibody and a 4-1BB (CD137) agonist.

As used herein, “combination” (and grammatical variations thereof such as “combine” or “combining”) encompasses combinations of an anti-HLA-G/anti-CD3 bispecific antibody and 4-1BB (CD137) agonist according to the invention wherein the anti-HLA-G/anti-CD3 bispecific antibody and the 4-1BB (CD137) agonist are in the same or in different containers, in the same or in different pharmaceutical formulations, administered together or separately, administered simultaneously or sequentially, in any order, and administered by the same or by different routes, provided that the anti-HLA-G/anti-CD3 bispecific antibody and the 4-1BB (CD137) agonist can simultaneously exert their biological effects in the body. For example “combining” an anti-HLA-G/anti-CD3 bispecific antibody and a 4-1BB (CD137) agonist according to the invention may mean first administering the anti-HLA-G/anti-CD3 bispecific antibody in a particular pharmaceutical formulation, followed by administration of the 4-1BB (CD137) agonist in another pharmaceutical formulation, or vice versa.

The anti-HLA-G/anti-CD3 bispecific antibody and the 4-1BB (CD137) agonist may be administered in any suitable manner known in the art. In one aspect, the anti-HLA-G/anti-CD3 bispecific antibody and the 4-1BB (CD137) agonist are administered sequentially (at different times). In another aspect, the anti-HLA-G/anti-CD3 bispecific antibody and the 4-1BB (CD137) agonist are administered concurrently (at the same time). Without wishing to be bound by theory, it may be advantageous to administer the 4-1BB (CD137) agonist prior to and/or concurrently with the anti-HLA-G/anti-CD3 bispecific antibody. In some aspects, the anti-HLA-G/anti-CD3 bispecific antibody is in a separate composition as the 4-1BB (CD137) agonist. In some aspects, the anti-HLA-G/anti-CD3 bispecific antibody is in the same composition as the 4-1BB (CD137) agonist.

The anti-HLA-G/anti-CD3 bispecific antibody and the 4-1BB (CD137) agonist can be administered by any suitable route, and may be administered by the same route of administration or by different routes of administration. In some aspects, the anti-HLA-G/anti-CD3 bispecific antibody is administered intravenously, intramuscularly, subcutaneously, topically, orally, transdermally, intraperitoneally, intraorbitally, by implantation, by inhalation, intrathecally, intraventricularly, or intranasally. In a particular aspect, the anti-HLA-G/anti-CD3 bispecific antibody is administrered intravenously. In some aspects, the 4-1BB (CD137) agonist is administered intravenously, intramuscularly, subcutaneously, topically, orally, transdermally, intraperitoneally, intraorbitally, by implantation, by inhalation, intrathecally, intraventricularly, or intranasally. In a particular aspect, the 4-1BB (CD137) agonist is administered intravenously. An effective amount of the anti-HLA-G/anti-CD3 bispecific antibody and the 4-1BB (CD137) agonist may be administered for prevention or treatment of disease. The appropriate route of administration and dosage of the anti-HLA-G/anti-CD3 bispecific antibody and/or the 4-1BB (CD137) agonist may be determined based on the type of disease to be treated, the type of the anti-HLA-G/anti-CD3 bispecific antibody, the type of 4-1BB (CD137) agonist, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. The anti-HLA-G/anti-CD3 bispecific antibody and the 4-1BB (CD137) agonist are suitably administered to the patient at one time or over a series of treatments.

Combinations of the invention can be used either alone or together with other agents in a therapy. For instance, a combination of the invention may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis. Combinations of the invention can also be combined with radiation therapy.

A kit as provided herein typically comprises one or more container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example, the container may be an intravenous solution bag or a vial having a stopper pierceable by a hypodermic injection needle). At least one active agent in the composition is an anti-HLA-G/anti-CD3 bispecific antibody to be used in the combinations of the invention. Another active agent is the 4-1BB (CD137) agonist to be used in the combinations of the invention, which may be in the same composition and container like the bispecific antibody, or may be provided in a different composition and container. The label or package insert indicates that the composition(s) is/are used for treating the condition of choice, such as cancer.

In one aspect, the invention provides a kit intended for the treatment of cancer, comprising in the same or in separate containers (a) an anti-HLA-G/anti-CD3 bispecific antibody, and (b) a 4-1BB (CD137) agonist, and optionally further comprising (c) a package insert comprising printed instructions directing the use of the combined treatment as a method for treating cancer. Moreover, the kit may comprise (a) a first container with a composition contained therein, wherein the composition comprises an anti-HLA-G/anti-CD3 bispecific antibody; (b) a second container with a composition contained therein, wherein the composition comprises a 4-1BB (CD137) agonist; and optionally (c) a third container with a composition contained therein, wherein the composition comprises a further cytotoxic or otherwise therapeutic agent. The kit in these aspects of the invention may further comprise a package insert indicating that the compositions can be used to treat cancer. Alternatively, or additionally, the kit may further comprise a third (or fourth) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Amino Acid Sequences

| SEQ | | |
|---|---|---|
| ID | | |
| NO: | Name | Sequence |
| 1 | Heavy chain CDR- | SYAMN |
| | H1, CD3-035-093 | |
| 2 | Heavy chain CDR- | RIRSKYNNYA TYYADSVKG |
| | H2, CD3-035-093 | |
| 3 | Heavy chain CDR- | ASNFPASYVS YFAY |
| | H3, CD3-035-093 | |
| 4 | Light chain CDR- | GSSTGAVTTS NYAN |
| | L1, CD3-035-093 | |
| 5 | Light chain CDR- | GTNKRAP |
| | L2, CD3-035-093 | |
| 6 | Light chain CDR- | ALWYSNLWV |
| | L3, CD3-035-093 | |
| 7 | Heavy chain variable | EVQLLESGGG LVQPGGSLRL SCAASGFTFS SYAMNWVRQA |
| | domain VH, CD3- | PGKGLEWVSR IRSKYNNYAT YYADSVKGRF TISRDDSKNT |
| | 035-093 | LYLQMNSLRA EDTAVYYCVR ASNFPASYVS YFAYWGQGTL |
| | | VTVSS |
| 8 | Light chain variable | QAVVTQEPSL TVSPGGTVTL TCGSSTGAVT TSNYANWVQE |
| | domain VL, CD3- | KPGQAFRGLI GGTNKRAPGT PARFSGSLLG GKAALTLSGA |
| | 035-093 | QPEDEAEYYC ALWYSNLWVF GGGTKLTVL |
| 9 | Heavy chain CDR- | SNRAAWN |
| | H1 HLAG-0090- | |
| | VL-S32P | |
| 10 | Heavy chain CDR- | RTYYRSKWYN DYAVSVQG |
| | H2 HLAG-0090- | |
| | VL-S32P | |
| 11 | Heavy chain CDR- | VRAVAPFDY |
| | H3 HLAG-0090- | |
| | VL-S32P | |
| 12 | Light chain CDR-L1 | KSSQSVLNPS NNKNNLA |
| | HLAG-0090-VL- | |
| | S32P | |
| 13 | Light chain CDR-L2 | WASTRES |
| | HLAG-0090-VL- | |
| | S32P | |
| 14 | Light chain CDR-L3 | QQYYRTPWT |
| | HLAG-0090-VL- | |
| | S32P | |
| 15 | Heavy chain variable | QVQLQQSGPG LLKPSQTLSL TCAISGDSVS SNRAAWNWIR |
| | domain VH, HLAG- | QSPSRGLEWL GRTYYRSKWY NDYAVSVQGR ITLIPDTSKN |
| | 0090-VL-S32P | QFSLRLNSVT PEDTAVYYCA SVRAVAPFDY WGQGVLVTVS |
| | | S |
| 16 | Light chain variable | DIVMTQSPDS LAVSLGERAT INCKSSQSVL NPSNNKNNLA |
| | domain VL, HLAG- | WYQQQPGQPP KLLIYWASTR ESGVPDRESG SGSGTDETLT |
| | 0090-VL-S32P | ISSLQAEDVA VYFCQQYYRT PWTFGQGTKV EIK |
| 17 | Heavy chain CDR- | SYAMS |
| | H1 FAP(4B9) | |
| 18 | Heavy chain CDR- | AIIGSGASTY YADSVKG |
| | H2 FAP(4B9) | |
| 19 | Heavy chain CDR- | GWFGGFNY |
| | H3 FAP(4B9) | |
| 20 | Light chain CDR-L1 | RASQSVTSSY LA |
| | FAP(4B9) | |
| 21 | Light chain CDR-L2 | VGSRRAT |
| | FAP(4B9) | |
| 22 | Light chain CDR-L3 | QQGIMLPPT |
| | FAP(4B9) | |
| 23 | Heavy chain variable | EVQLLESGGG LVQPGGSLRL SCAASGFTFS SYAMSWVRQA |
| | domain VH, | PGKGLEWVSA IIGSGASTYY ADSVKGRFTI SRDNSKNTLY |
| | FAP(4B9) | LQMNSLRAED TAVYYCAKGW FGGFNYWGQG TLVTVSS |
| 24 | Light chain variable | EIVLTQSPGT LSLSPGERAT LSCRASQSVT SSYLAWYQQK |
| | domain VL, | PGQAPRLLIN VGSRRATGIP DRFSGSGSGT DFTLTISRLE |
| | FAP(4B9) | PEDFAVYYCQ QGIMLPPTFG QGTKVEIK |
| 25 | Human (hu) 4-1 | REGPELSPDD PAGLLDLRQG MFAQLVAQNV LLIDGPLSWY |
| | BBL (71-254) | SDPGLAGVSL TGGLSYKEDT KELVVAKAGV YYVFFQLELR |
| | | RVVAGEGSGS VSLALHLQPL RSAAGAAALA LTVDLPPASS |
| | | EARNSAFGFQ GRLLHLSAGQ RLGVHLHTEA RARHAWQLTQ |
| | | GATVLGLFRV TPEIPAGLPS PRSE |
| 26 | Human (hu) 4-1 | LDLRQGMFAQ LVAQNVLLID GPLSWYSDPG LAGVSLTGGL |
| | BBL (85-254) | SYKEDTKELV VAKAGVYYVF FQLELRRVVA GEGSGSVSLA |
| | | LHLQPLRSAA GAAALALTVD LPPASSEARN SAFGFQGRLL |
| | | HLSAGQRLGV HLHTEARARH AWQLTQGATV LGLFRVTPEI |
| | | PAGLPSPRSE |
| 27 | Human (hu) 4-1BBL | DPAGLLDLRQ GMFAQLVAQN VLLIDGPLSW YSDPGLAGVS |
| | (80-254) | LTGGLSYKED TKELVVAKAG VYYVFFQLEL RRVVAGEGSG |
| | | SVSLALHLQP LRSAAGAAAL ALTVDLPPAS SEARNSAFGE |
| | | QGRLLHLSAG QRLGVHLHTE ARARHAWQLT QGATVLGLER |
| | | VTPEIPAGLP SPRSE |
| 28 | Human (hu) 4-1BBL | PWAVSGARAS PGSAASPRLR EGPELSPDDP AGLLDLRQGM |
| | (52-254) | FAQLVAQNVL LIDGPLSWYS DPGLAGVSLT GGLSYKEDTK |
| | | ELVVAKAGVY YVFFQLELRR VVAGEGSGSV SLALHLQPLR |
| | | SAAGAAALAL TVDLPPASSE ARNSAFGFQG RLLHLSAGQR |
| | | LGVHLHTEAR ARHAWQLTQG ATVLGLFRVT PEIPAGLPSP |
| | | RSE |
| 29 | Human (hu) 4-1BBL | REGPELSPDD PAGLLDLRQG MFAQLVAQNV LLIDGPLSWY |
| | (71-248) | SDPGLAGVSL TGGLSYKEDT KELVVAKAGV YYVFFQLELR |
| | | RVVAGEGSGS VSLALHLQPL RSAAGAAALA LTVDLPPASS |
| | | EARNSAFGFQ GRLLHLSAGQ RLGVHLHTEA RARHAWQLTQ |
| | | GATVLGLFRV TPEIPAGL |
| 30 | Human (hu) 4-1BBL | LDLRQGMFAQ LVAQNVLLID GPLSWYSDPG LAGVSLTGGL |
| | (85-248) | SYKEDTKELV VAKAGVYYVF FQLELRRVVA GEGSGSVSLA |
| | | LHLQPLRSAA GAAALALTVD LPPASSEARN SAFGFQGRLL |
| | | HLSAGQRLGV HLHTEARARH AWQLTQGATV LGLERVTPEI |
| | | PAGL |
| 31 | Human (hu) 4-1BBL | DPAGLLDLRQ GMFAQLVAQN VLLIDGPLSW YSDPGLAGVS |
| | (80-248) | LTGGLSYKED TKELVVAKAG VYYVFFQLEL RRVVAGEGSG |
| | | SVSLALHLQP LRSAAGAAAL ALTVDLPPAS SEARNSAFGE |
| | | QGRLLHLSAG QRLGVHLHTE ARARHAWQLT QGATVLGLER |
| | | VTPEIPAGL |
| 32 | Human (hu) 4-1BBL | PWAVSGARAS PGSAASPRLR EGPELSPDDP AGLLDLRQGM |
| | (52-248) | FAQLVAQNVL LIDGPLSWYS DPGLAGVSLT GGLSYKEDTK |
| | | ELVVAKAGVY YVFFQLELRR VVAGEGSGSV SLALHLQPLR |
| | | SAAGAAALAL TVDLPPASSE ARNSAFGFQG RLLHLSAGQR |
| | | LGVHLHTEAR ARHAWQLTQG ATVLGLERVT PEIPAGL |
| 33 | dimeric hu 4-1BBL | REGPELSPDD PAGLLDLRQG MFAQLVAQNV LLIDGPLSWY |
| | (71-254) connected | SDPGLAGVSL TGGLSYKEDT KELVVAKAGV YYVFFQLELR |
| | by (G₄S)₂ linker | RVVAGEGSGS VSLALHLQPL RSAAGAAALA LTVDLPPASS |
| | | EARNSAFGFQ GRLLHLSAGQ RLGVHLHTEA RARHAWQLTQ |
| | | GATVLGLFRV TPEIPAGLPS PRSEGGGGSG GGGSREGPEL |
| | | SPDDPAGLLD LRQGMFAQLV AQNVLLIDGP LSWYSDPGLA |
| | | GVSLTGGLSY KEDTKELVVA KAGVYYVFFQ LELRRVVAGE |
| | | GSGSVSLALH LQPLRSAAGA AALALTVDLP PASSEARNSA |
| | | FGFQGRLLHL SAGQRLGVHL HTEARARHAW QLTQGATVLG |
| | | LERVTPEIPA GLPSPRSE |
| 34 | dimeric hu 4-1BBL | LDLRQGMFAQ LVAQNVLLID GPLSWYSDPG LAGVSLTGGL |
| | (85-254) connected | SYKEDTKELV VAKAGVYYVF FQLELRRVVA GEGSGSVSLA |
| | by (G₄S)₂ linker | LHLQPLRSAA GAAALALTVD LPPASSEARN SAFGFQGRLL |
| | | HLSAGQRLGV HLHTEARARH AWQLTQGATV LGLFRVTPEI |
| | | PAGLPSPRSE GGGGSGGGGS LDLRQGMFAQ LVAQNVLLID |
| | | GPLSWYSDPG LAGVSLTGGL SYKEDTKELV VAKAGVYYVE |
| | | FQLELRRVVA GEGSGSVSLA LHLQPLRSAA GAAALALTVD |
| | | LPPASSEARN SAFGFQGRLL HLSAGQRLGV HLHTEARARH |
| | | AWQLTQGATV LGLERVTPEI PAGLPSPRSE |
| 35 | dimeric hu 4-1BBL | DPAGLLDLRQ GMFAQLVAQN VLLIDGPLSW YSDPGLAGVS |
| | (80-254) connected | LTGGLSYKED TKELVVAKAG VYYVFFQLEL RRVVAGEGSG |
| | by (G₄S)₂ linker | SVSLALHLQP LRSAAGAAAL ALTVDLPPAS SEARNSAFGE |
| | | QGRLLHLSAG QRLGVHLHTE ARARHAWQLT QGATVLGLER |
| | | VTPEIPAGLP SPRSEGGGGS GGGGSDPAGL LDLRQGMFAQ |
| | | LVAQNVLLID GPLSWYSDPG LAGVSLTGGI SYKEDTKELV |
| | | VAKAGVYYVF FQLELRRVVA GEGSGSVSLA LHLQPLRSAA |
| | | GAAALALTVD LPPASSEARN SAFGFQGRLL HLSAGQRLGV |
| | | HLHTEARARH AWQLTQGATV LGLFRVTPEI PAGLPSPRSE |
| 36 | dimeric hu 4-1BBL | PWAVSGARAS PGSAASPRLR EGPELSPDDP AGLLDLRQGM |
| | (52-254) connected | FAQLVAQNVL LIDGPLSWYS DPGLAGVSLT GGLSYKEDTK |
| | by (G₄S)₂ linker | ELVVAKAGVY YVFFQLELRR VVAGEGSGSV SLALHLQPLR |
| | | SAAGAAALAL TVDLPPASSE ARNSAFGFQG RLLHLSAGQR |
| | | LGVHLHTEAR ARHAWQLTQG ATVLGLERVT PEIPAGLPSP |
| | | RSEGGGGSGG GGSPWAVSGA RASPGSAASP RLREGPELSP |
| | | DDPAGLLDLR QGMFAQLVAQ NVLLIDGPLS WYSDPGLAGV |
| | | SLTGGLSYKE DTKELVVAKA GVYYVFFQLE LRRVVAGEGS |
| | | GSVSLALHLQ PLRSAAGAAA LALTVDLPPA SSEARNSAFG |
| | | FQGRLLHLSA GQRLGVHLHT EARARHAWQL TQGATVLLF |
| | | RVTPEIPAGL PSPRSE |
| 37 | dimeric hu 4-1BBL | REGPELSPDD PAGLLDLRQG MFAQLVAQNV LLIDGPLSWY |
| | (71-248) connected | SDPGLAGVSL TGGLSYKEDT KELVVAKAGV YYVFFQLELR |
| | by (G₄S)₂ linker | RVVAGEGSGS VSLALHLQPL RSAAGAAALA LTVDLPPASS |
| | | EARNSAFGFQ GRLLHLSAGQ RLGVHLHTEA RARHAWQLTQ |
| | | GATVLGLFRV TPEIPAGLGG GGSGGGGSRE GPELSPDDPA |
| | | GLLDLRQGMF AQLVAQNVLL IDGPLSWYSD PGLAGVSLTG |
| | | GLSYKEDTKE LVVAKAGVYY VFFQLELRRV VAGEGSGSVS |
| | | LALHLQPLRS AAGAAALALT VDLPPASSEA RNSAFGFQGR |
| | | LLHLSAGQRL GVHLHTEARA RHAWQLTQGA TVLGLERVTP |
| | | EIPAGL |
| 38 | dimeric hu 4-1BBL | LDLRQGMFAQ LVAQNVLLID GPLSWYSDPG LAGVSLTGGL |
| | (85-248) connected | SYKEDTKELV VAKAGVYYVF FQLELRRVVA GEGSGSVSLA |
| | by (G₄S)₂ linker | LHLQPLRSAA GAAALALTVD LPPASSEARN SAFGFQGRLL |
| | | HLSAGQRLGV HLHTEARARH AWQLTQGATV LGLERVTPEI |
| | | PAGLGGGGSG GGGSLDLRQG MFAQLVAQNV LLIDGPLSWY |
| | | SDPGLAGVSL TGGLSYKEDT KELVVAKAGV YYVFFQLELR |
| | | RVVAGEGSGS VSLALHLQPL RSAAGAAALA LTVDLPPASS |
| | | EARNSAFGFQ GRLLHLSAGQ RLGVHLHTEA RARHAWQLTQ |
| | | GATVLGLFRV TPEIPAGL |
| 39 | dimeric hu 4-1BBL | DPAGLLDLRQ GMFAQLVAQN VLLIDGPLSW YSDPGLAGVS |
| | (80-248) connected | LTGGLSYKED TKELVVAKAG VYYVFFQLEL RVVAGEGSGS |
| | by (G₄S)₂ linker | SVSLALHLQP LRSAAGAAAL ALTVDLPPAS SEARNSAFGE |
| | | QGRLLHLSAG QRLGVHLHTE ARARHAWQLT QGATVLGLER |
| | | VTPEIPAGLG GGGSGGGGSD PAGLLDLRQG MFAQLVAQNV |
| | | LLIDGPLSWY SDPGLAGVSL TGGLSYKEDT KELVVAKAGV |
| | | YYVFFQLELR RRVVAGEGSG VSLALHLQPL RSAAGAAALA |
| | | LTVDLPPASS EARNSAFGFQ GRLLHLSAGQ RLGVHLHTEA |
| | | RARHAWQLTQ GATVLGLERV TPEIPAGL |
| 40 | dimeric hu 4-1BBL | PWAVSGARAS PGSAASPRLR EGPELSPDDP AGLLDLRQGM |
| | (52-248) connected | FAQLVAQNVL LIDGPLSWYS DPGLAGVSLT GGLSYKEDTK |
| | by (G₄S)₂ linker | ELVVAKAGVY YVFFQLELRR VVAGEGSGSV SLALHLQPLR |
| | | SAAGAAALAL TVDLPPASSE ARNSAFGFQG RLLHLSAGQR |
| | | LGVHLHTEAR ARHAWQLTQG ATVLGLFRVT PEIPAGLGGG |
| | | GSGGGGSPWA VSGARASPGS AASPRLREGP ELSPDDPAGL |
| | | LDLRQGMFAQ LVAQNVLLID GPLSWYSDPG LAGVSLTGGL |
| | | SYKEDTKELV VAKAGVYYVF FQLELRRVVA GEGSGSVSLA |
| | | LHLQPLRSAA GAAALALTVD LPPASSEARN SAFGFQGRLL |
| | | HLSAGQRLGV HLHTEARARH AWQLTQGATV LGLFRVTPEI |
| | | PAGL |
| 41 | human leukocyte | GSHSMRYFSA AVSRPGRGEP RFIAMGYVDD TQFVREDSDS |
| | antigen G (HLA-G) | ACPRMEPRAP WVEQEGPEYW EEETRNTKAH AQTDRMNLQT |
| | exemplary sequence | LRGYYNQSEA SSHTLQWMIG CDLGSDGRLL RGYEQYAYDG |
| | | KDYLALNEDL RSWTAADTAA QISKRKCEAA NVAEQRRAYL |
| | | EGTCVEWLHR YLENGKEMLQ RADPPKTHVT HHPVEDYEAT |
| | | LRCWALGFYP AEIILTWQRD GEDQTQDVEL VETRPAGDGT |
| | | FQKWAAVVVP SGEEQRYTCH VQHEGLPEPL MLRWKQSSLP |
| | | TIPIMGIVAG LVVLAAVVTG AAVAAVLWRK KSSD |
| 42 | HLA-G β2M MHC I | RIIPRHLQLG CGGSGGGGSG GGGSIQRTPK IQVYSRHPAE |
| | complex exemplary | NGKSNFLNCY VSGFHPSDIE VDLLKNGERI EKVEHSDLSF |
| | sequence | SKDWSFYLLY YTEFTPTEKD EYACRVNHVT LSQPKIVKWD |
| | | RDMGGGGSGG GGSGGGGSGG GGSGSHSMRY FSAAVSRPGR |
| | | GEPRFIAMGY VDDTQFVRED SDSACPRMEP RAPWVEQEGP |
| | | EYWEEETRNT KAHAQTDRMN LQTLRGCYNQ SEASSHTLQW |
| | | MIGCDLGSDG RLLRGYEQYA YDGKDYLALN EDLRSWTAAD |
| | | TAAQISKRKC EAANVAEQRR AYLEGTCVEW LHRYLENGKE |
| | | MLQRADPPKT HVTHHPVEDY EATLRCWALG FYPAEIILTW |
| | | QRDGEDQTQD VELVETRPAG DGTFQKWAAV VVPSGEEQRY |
| | | TCHVQHEGLP EPLMLRWGSG LNDIFEAQKI EWHEHHHHHH |
| 43 | human IgG1 Fc | DKTHTCPPCP APELLGGPSV FLFPPKPKDT LMISRTPEVT |
| | region exemplary | CVVVDVSHED PEVKFNWYVD GVEVHNAKTK PREEQYNSTY |
| | sequence | RVVSVLTVLH QDWLNGKEYK CKVSNKALPA PIEKTISKAK |
| | | GQPREPQVYT LPPSRDELTK NQVSLTCLVK GFYPSDIAVE |
| | | WESNGQPENN YKTTPPVLDS DGSFFLYSKL TVDKSRWQQG |
| | | NVFSCSVMHE ALHNHYTQKS LSLSP |
| 44 | human CD3 | MQSGTHWRVL GLCLLSVGVW GQDGNEEMGG ITQTPYKVSI |
| | | SGTTVILTCP QYPGSEILWQ HNDKNIGGDE DDKNIGSDED |
| | | HLSLKEFSEL EQSGYYVCYP RGSKPEDANF YLYLRARVCE |
| | | NCMEMDVMSV ATIVIVDICI TGGLLLLVYY WSKNRKAKAK |
| | | PVTRGAGAGG RQRGQNKERP PPVPNPDYEP IRKGQRDLYS |
| | | GLNQRRI |
| 45 | cynomolgus CD3 | MQSGTRWRVL GLCLLSIGVW GQDGNEEMGS ITQTPYQVSI |
| | | SGTTVILTCS QHLGSEAQWQ HNGKNKEDSG DRLFLPEFSE |
| | | MEQSGYYVCY PRGSNPEDAS HHLYLKARVC ENCMEMDVMA |
| | | VATIVIVDIC ITLGLLLLVY YWSKNRKAKA KPVTRGAGAG |
| | | GRQRGQNKER PPPVPNPDYE PIRKGQQDLY SGLNQRRI |
| 46 | HLA-G-VL-CL(RK) | DIVMTQSPDS LAVSLGERAT INCKSSQSVL NPSNNKNNLA |
| | | WYQQQPGQPP KLLIYWASTR ESGVPDRESG SGSGTDETLT |
| | | ISSLQAEDVA VYFCQQYYRT PWTFGQGTKV EIKRTVAAPS |
| | | VFIFPPSDRK LKSGTASVVC LLNNFYPREA KVQWKVDNAL |
| | | QSGNSQESVT EQDSKDSTYS LSSTLTLSKA DYEKHKVYAC |
| | | EVTHQGLSSP VTKSFNRGEC |
| 47 | HLA-G-VH- | QVQLQQSGPG LLKPSQTLSL TCAISGDSVS SNRAAWNWIR |
| | CH1(EE)-Fc(hole, | QSPSRGLEWL GRTYYRSKWY NDYAVSVQGR ITLIPDTSKN |
| | PGLALA) | QFSLRLNSVT PEDTAVYYCA SVRAVAPEDY WGQGVLVTVS |
| | | SASTKGPSVF PLAPSSKSTS GGTAALGCLV EDYFPEPVTV |
| | | SWNSGALTSG VHTFPAVLQS SGLYSLSSVV TVPSSSLGTQ |
| | | TYICNVNHKP SNTKVDEKVE PKSCDKTHTC PPCPAPEAAG |
| | | GPSVFLFPPK PKDTLMISRT PEVTCVVVDV SHEDPEVKFN |
| | | WYVDGVEVHN AKTKPREEQY NSTYRVVSVL TVLHQDWLNG |
| | | KEYKCKVSNK ALGAPIEKTI SKAKGQPREP QVCTLPPSRD |
| | | ELTKNQVSLS CAVKGFYPSD IAVEWESNGQ PENNYKTTPP |
| | | VLDSDGSFFL VSKLTVDKSR WQQGNVFSCS VMHEALHNHY |
| | | TQKSLSLSPG K |
| 48 | CD3 VH-CL | EVQLLESGGG LVQPGGSLRL SCAASGFTFS SYAMNWVRQA |
| | | PGKGLEWVSR IRSKYNNYAT YYADSVKGRF TISRDDSKNT |
| | | LYLQMNSLRA EDTAVYYCVR ASNFPASYVS YFAYWGQGTL |
| | | VTVSSASVAA PSVFIFPPSD EQLKSGTASV VCLLNNFYPR |
| | | EAKVQWKVDN ALQSGNSQES VTEQDSKDST YSLSSTLTLS |
| | | KADYEKHKVY ACEVTHQGLS SPVTKSFNRG EC |
| 49 | HLA-G VH- | QVQLQQSGPG LLKPSQTLSL TCAISGDSVS SNRAAWNWIR |
| | CH1(EE)-CD3 VL- | QSPSRGLEWL GRTYYRSKWY NDYAVSVQGR ITLIPDTSKN |
| | CH1-Fc(knob, | QFSLRLNSVT PEDTAVYYCA SVRAVAPEDY WGQGVLVTVS |
| | PGLALA) | SASTKGPSVF PLAPSSKSTS GGTAALGCLV EDYFPEPVTV |
| | | SWNSGALTSG VHTFPAVLQS SGLYSLSSVV TVPSSSLGTQ |
| | | TYICNVNHKP SNTKVDEKVE PKSCDGGGGS GGGGGQAVVT |
| | | QEPSLTVSPG GTVTLTCGSS TGAVTTSNYA NWVQEKPGQA |
| | | FRGLIGGTNK RAPGTPARFS GSLLGGKAAL TLSGAQPEDE |
| | | AEYYCALWYS NLWVFGGGTK LTVLSSASTK GPSVFPLAPS |
| | | SKSTSGGTAA LGCLVKDYFP EPVTVSWNSG ALTSGVHTFP |
| | | AVLQSSGLYS LSSVVTVPSS SLGTQTYICN VNHKPSNTKV |
| | | DKKVEPKSCD KTHTCPPCPA PEAAGGPSVF LFPPKPKDTL |
| | | MISRTPEVTC VVVDVSHEDP EVKFNWYVDG VEVHNAKTKP |
| | | REEQYNSTYR VVSVLTVLHQ DWLNGKEYKC KVSNKALGAP |
| | | IEKTISKAKG QPREPQVYTL PPCRDELTKN QVSLWCLVKG |
| | | FYPSDIAVEW ESNGQPENNY KTTPPVLDSD GSFFLYSKLT |
| | | VDKSRWQQGN VFSCSVMHEA LHNHYTQKSL SLSPGK |
| 50 | hu 4-1BBL (71- | REGPELSPDD PAGLLDLRQG MFAQLVAQNV LLIDGPLSWY |
| | 248)-(G₄S)₂-hu 4- | SDPGLAGVSL TGGLSYKEDT KELVVAKAGV YYVFFQLELR |
| | 1BBL (71-248) | RVVAGEGSGS VSLALHLQPL RSAAGAAALA LTVDLPPASS |
| | | EARNSAFGFQ GRLLHLSAGQ RLGVHLHTEA RARHAWQLTQ |
| | | GATVLGLFRV TPEIPAGLGG GGSGGGGSRE GPELSPDDPA |
| | | GLLDLRQGMF AQLVAQNVLL IDGPLSWYSD PGLAGVSLTG |
| | | GLSYKEDTKE LVVAKAGVYY VFFQLELRRV VAGEGSGSVS |
| | | LALHLQPLRS AAGAAALALT VDLPPASSEA RNSAFGFQGR |
| | | LLHLSAGQRL GVHLHTEARA RHAWQLTQGA TVLGLFRVTP |
| | | EIPAGL |
| 51 | hu 4-1BBL (71- | REGPELSPDD PAGLLDLRQG MFAQLVAQNV LLIDGPLSWY |
| | 248)-(G₄S)₂-hu 4- | SDPGLAGVSL TGGLSYKEDT KELVVAKAGV YYVFFQLELR |
| | 1BBL (71-248)- | RVVAGEGSGS VSLALHLQPL RSAAGAAALA LTVDLPPASS |
| | CL(RK)-Fc(knob, | EARNSAFGFQ GRLLHLSAGQ RLGVHLHTEA RARHAWQLTQ |
| | PGLALA) | GATVLGLFRV TPEIPAGLGG GGSGGGGSRE GPELSPDDPA |
| | | GLLDLRQGMF AQLVAQNVLL IDGPLSWYSD PGLAGVSLTG |
| | | GLSYKEDTKE LVVAKAGVYY VFFQLELRRV VAGEGSGSVS |
| | | LALHLQPLRS AAGAAALALT VDLPPASSEA RNSAFGFQGR |
| | | LLHLSAGQRL GVHLHTEARA RHAWQLTQGA TVLGLFRVTP |
| | | EIPAGLGGGG SGGGGSRTVA APSVFIFPPS DRKLKSGTAS |
| | | VVCLLNNFYP REAKVQWKVD NALQSGNSQE SVTEQDSKDS |
| | | TYSLSSTLTL SKADYEKHKV YACEVTHQGL SSPVTKSENR |
| | | GECDKTHTCP PCPAPEAAGG PSVFLFPPKP KDTLMISRTP |
| | | EVTCVVVDVS HEDPEVKFNW YVDGVEVHNA KTKPREEQYN |
| | | STYRVVSVLT VLHQDWLNGK EYKCKVSNKA LGAPIEKTIS |
| | | KAKGQPREPQ VYTLPPCRDE LTKNQVSLWC LVKGFYPSDI |
| | | AVEWESNGQP ENNYKTTPPV LDSDGSFFLY SKLTVDKSRW |
| | | QQGNVFSCSV MHEALHNHYT QKSLSLSP |
| 52 | hu 4-1BBL (71- | REGPELSPDD VSLALHLQPL RSAAGAAALA LLIDGPLSWY |
| | 248)-CH1(EE) | SDPGLAGVSL PAGLLDLRQG MFAQLVAQNV YYVFFQLELR |
| | | RVVAGEGSGS TGGLSYKEDT KELVVAKAGV LTVDLPPASS |
| | | EARNSAFGFQ GRLLHLSAGQ RLGVHLHTEA RARHAWQLTQ |
| | | GATVLGLFRV TPEIPAGLGG GGSGGGGSAS TKGPSVFPLA |
| | | PSSKSTSGGT AALGCLVEDY FPEPVTVSWN SGALTSGVHT |
| | | FPAVLQSSGL YSLSSVVTVP SSSLGTQTYI CNVNHKPSNT |
| | | KVDEKVEPKS C |
| 53 | FAP VH-CH1- | EVQLLESGGG LVQPGGSLRL SCAASGFTFS SYAMSWVRQA |
| | Fc(hole, PGLALA) | PGKGLEWVSA IIGSGASTYY ADSVKGRFTI SRDNSKNTLY |
| | | LQMNSLRAED TAVYYCAKGW FGGFNYWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APEAAGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALGA PIEKTISKAK GQPREPQVCT LPPSRDELTK |
| | | NQVSLSCAVK GFYPSDIAVE WESNGQPENN YKTTPPVLDS |
| | | DGSFFLVSKL TVDKSRWQQG NVFSCSVMHE ALHNHYTQKS |
| | | LSLSP |
| 54 | FAP VL-CL | EIVLTQSPGT LSLSPGERAT LSCRASQSVT SSYLAWYQQK |
| | | PGQAPRLLIN VGSRRATGIP DRFSGSGSGT DFTLTISRLE |
| | | PEDFAVYYCQ QGIMLPPTFG QGTKVEIKRT VAAPSVFIFP |
| | | PSDEQLKSGT ASVVCLLNNF YPREAKVQWK VDNALQSGNS |
| | | QESVTEQDSK DSTYSLSSTL TLSKADYEKH KVYACEVTHQ |
| | | GLSSPVTKSF NRGEC |
| 55 | hu 4-1BBL (50-254) | ACPWAVSGAR ASPGSAASPR LREGPELSPD DPAGLLDLRQ |
| | | GMFAQLVAQN VLLIDGPLSW YSDPGLAGVS LTGGLSYKED |
| | | TKELVVAKAG VYYVFFQLEL RRVVAGEGSG SVSLALHLQP |
| | | LRSAAGAAAL ALTVDLPPAS SEARNSAFGF QGRLLHLSAG |
| | | QRLGVHLHTE ARARHAWQLT QGATVLGLFR VTPEIPAGLP |
| | | SPRSE |
| 56 | Peptide linker G₄S | GGGGS |
| 57 | Peptide linker (G₄S)₂ | GGGGSGGGGS |
| 58 | Peptide linker (G₄S)₃ | GGGGSGGGGSGGGGS |
| 59 | Peptide linker (G₄S)₄ | GGGGSGGGGSGGGGSGGGGS |
| 60 | Peptide linker | GGGGS GGGGG |

### BRIEF DESCRIPTION OF THE DRAWINGS

FIG. 1A: Schematic illustration of the HLA-G-targeted T cell bispecific (TCB) antibody molecule used in the Examples (“HLA-G TCB”). The molecule comprises a single antigen-binding moiety for CD3, two antigen-binding moieties for HLA-G, and an Fc domain. FIG. 1B: Schematic illustration of the FAP-targeted 4-1BB (CD137) agonist used in the Examples (“FAP4-1BBL”). The molecule comprises a 4-1BBL ectodomain trimer, an antigen-binding moiety for FAP, and an Fc domain. Black dot: modification in the Fc domain promoting heterodimerization. *: amino acids of opposite charges introduced in the CH and CL domains.

FIG. 2 shows the protocol of the in vivo efficacy study of HLA-G TCB (P1AF7977) vs. HLA-G TCB (P1AF7977) in combination with FAP4-1BBL (P1AD5195) in BC004 PDX-bearing fully humanized NSG mice. The table underneath the treatment scheme shows which subgroups of mice received which treatments. Animals that obtained histidine buffer (vehicle) were used as negative control. After the tumors reached an average volume of around 200-300 mm³, mice were randomized to five groups receiving: A) histidine buffer (vehicle) as control; B) HLA-G-TCB (0.5 mg/kg once per week intravenously), C) HLA-G-TCB (0.05 mg/kg once per week intravenously), D) HLA-G-TCB (0.5 mg/kg once per week intravenously)+FAP4-1-BBL (1 mg/kg every 2 weeks intravenously), E) HLA-G-TCB (0.05 mg/kg once per week intravenously)+FAP4-1-BBL (1 mg/kg every 2 weeks intravenously). The experiment is described in Example 1.

FIGS. 3A and 3B show the efficacy of an anti-HLA-G/anti-CD3 bispecific antibody (P1AF7977), in two different doses (0.05 mg/kg and 0.5 mg/kg) either alone or in combination with FAP4-1BBL (at 1 mg/kg) in the BC004 PDX mouse model with FAP-expressing stroma. The Figures show tumor growth curves (represented as tumor volumes) of mice subjected to the indicated treatments (for the treatment scheme refer to FIG. 2). FIG. 3A shows an overlay of all data in a single graph, while FIG. 3B shows the data obtained for each animal, with separate graphs for the different treatment groups.

FIG. 4 shows a quantification of the cellular and molecular immune components associated with the anti-tumor effect of the treatments of the experiment described in FIG. 2. Flow cytometry analysis of the indicated immune populations in tumors at day 9 post randomization (i.e. 2 days after the second injection) was performed. The number of immune cells infiltrating the tumor is shown for CD45+ cells as percentage of viable cells and in absolute numbers (counts/mg). The number of CD3+ T cells and CD68+ macrophages is shown in absolute numbers (counts per mg) and as ratio per immune cells (CD45+).

FIG. 5 shows the protocol of the in vivo efficacy study of HLA-G TCB (P1AF7977) vs. HLA-G TCB (P1AF7977) in combination with FAP4-1BBL (P1AD5195) in CR5044-bearing fully humanized NSG mice. The table underneath the treatment scheme shows which of the subgroups of mice receiving which treatments/combinations. Animals that obtained histidine buffer (vehicle) were used as negative control. After the tumors reached an average volume of around 200-300 mm³, mice were randomized to 5 groups receiving: A) histidine buffer (vehicle) as control; B) HLA-G-TCB (2.5 mg/kg once per week intravenously), C) HLA-G-TCB (0.5 mg/kg once per week intravenously), D) HLA-G-TCB (0.1 mg/kg once per week intravenously), E) HLA-G-TCB (0.5 mg/kg once per week intravenously)+FAP4-1-BBL (1 mg/kg every 2 weeks, intravenously). The experiment is described in Example 2.

FIGS. 6A and 6B show the efficacy of a bispecific anti-HLA-G/anti-CD3 bispecific antibody (P1AF7977), in the CR5044 PDX mouse model with FAP-expressing stroma (FAP4-1BBL treatment), either alone in three different doses (0.1 mg/kg, 0.5 mg/kg and 2.5 mg/kg), or at a concentration of 0.5 mg/kg in combination with FAP4-1-BBL at 1 mg/kg. The figures show tumor growth curves (represented as tumor volumes) of mice subjected to the indicated treatments. FIG. 6A shows an overlay of all data in a single graph, while FIG. 6B shows the data obtained for each animal, with separate graphs for the different treatment schedules.

FIG. 7 shows a quantification of the cellular immune components associated with the anti-tumor effect of the treatments of the experiment described in FIG. 5. Flow cytometry analysis of the indicated immune populations in tumors was performed at day 9 post randomization (i.e. 2 days after the second injection). The number of immune cells is shown for CD45+ cells as percentage of viable cells and as absolute numbers (counts per mg). The number of CD4+ T cells, CD8+ T cells, Tregs (FoxP3+), NK cells (CD56+) and macrophages (CD68+) is shown in absolute numbers (counts per mg), and for macrophages also as percentage of immune cells (CD45+). Granzyme B (GZMB) expression on NK cells was shown as percent of CD56+ cells. Animals that received histidine buffer (vehicle) were used as negative control.

FIG. 8 shows a quantification of the soluble CD25 release-indicating T cell activation-associated with the anti-tumor effect of the treatments of the experiment described in FIG. 5. Analysis of the indicated soluble CD25 levels in serum samples of CR5044 tumor-bearing mice was performed at 6 hours post first and post fourth treatment cycle). Animals that obtained histidine buffer (vehicle) were used as negative control.

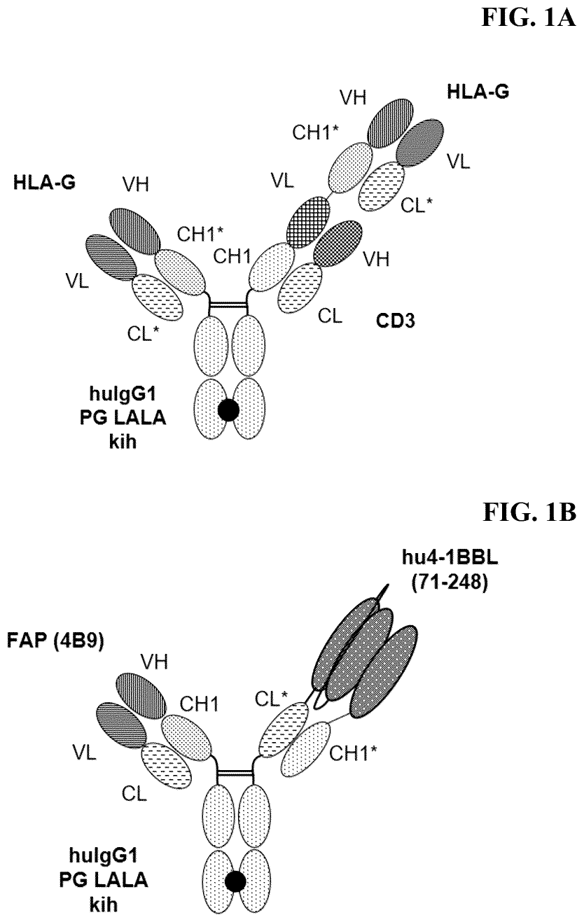

### EXAMPLES

The following are examples of methods and compositions of the invention. It is understood that various other aspects may be practiced, given the general description provided above.

### Example 1—Antitumor Activity of HLA-G TCB Monotherapy and Combination Therapy with FAP4-1BBL in the BC004 PDX Model in Humanized NSG Mice

To monitor tumor growth inhibition (TGI) induced by either anti-HLA-G/anti-CD3 bispecific antibody (in the following also referred to as “PIAF7977”) alone or a combination of anti-HLA-G/anti-CD3 bispecific antibody and FAP4-1BBL (in the following also referred to as “P1AD5195”), female 3-week-old NSG mice were humanized by irradiation (140 cGy) and intravenous injection with 9×10⁴ CD34+ cord blood cells/mouse at Jackson Laboratories. The sequences of the molecules used in the study are shown in the Table below. After reaching a human immune infiltrate (as indicated by human CD45+ cells) above 25% in blood, humanized NSG mice were shipped to Roche and maintained for 5 days to get accustomed to the new environment. Mice were kept under specific-pathogen-free conditions with daily cycles of 12 h light/12 h darkness according to committed guidelines (GV-Solas; Felasa; TierschG). Continuous health monitoring was carried out on a daily basis. The experimental study protocol was reviewed and approved by the local government (ROB-55.2-2532. Vet_03-16-10).

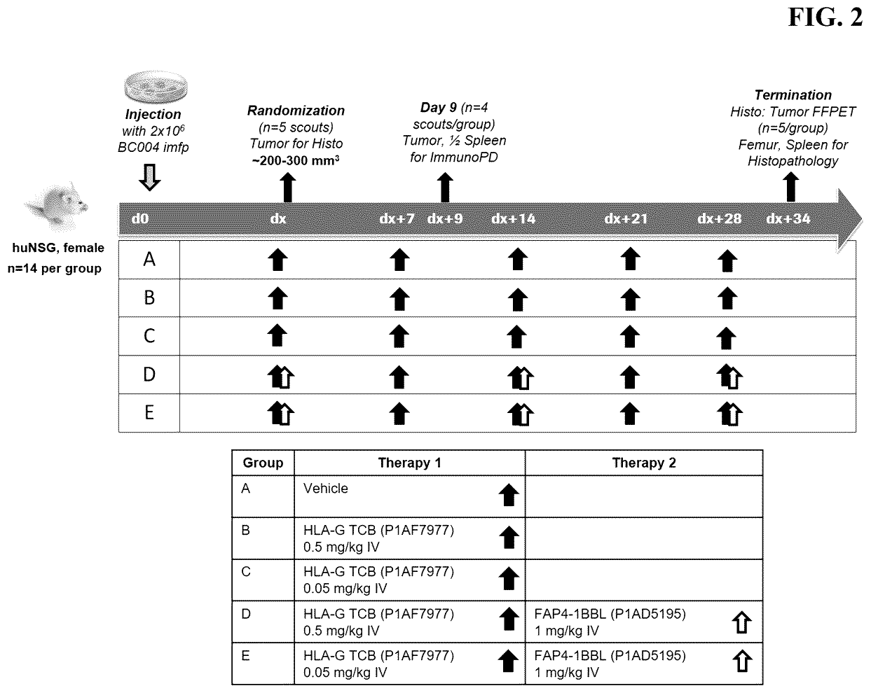

For the assay in the BC004 PDX model, the human breast cancer PDX model BC004 was purchased from OncoTest (Freiburg, Germany). Tumor fragments were digested with collagenase D (Catalog No. 11088882001; Sigma-Aldrich; Minneapolis, MI) and DNase I (Catalog No. 10104159001; Sigma-Aldrich) for 20 minutes to prepare single-cell suspensions. Cell number and viability were determined using a ViCell counter (Beckman Coulter; Brea, CA). On day zero (“d0”), humanized mice were injected in the intramammary fat pad with 2×10⁶ BC004 cells in a total volume of 20 μL phosphate-buffered saline. Once tumors reached an average volume of approximately 200-300 mm³ (=“dx”), mice were randomized into different treatment groups. The first group of mice received histidine buffer (vehicle) as control. All molecules were prepared freshly in 20 mM Histidine, 140 mM NaCl, pH 6.0 before injection and administered intravenously (IV) at the dose and schedule indicated in FIG. 2. HLA-G TCB was injected weekly and, if applicable, FAP4-1BBL was injected every 2 weeks.

TABLE
Amino acid sequences of the molecules used in the examples

| | | SEQ ID NO: | | | |
|---|---|---|---|---|---|
| | | 1st | 2nd | 3rd | 4th |
| | Molecule | poly- | poly- | poly- | poly- |
| Molecule | ID | peptide | pepide | peptide | peptide |
| HLA-G TCB | P1AF7977 | 46 | 47 | 48 | 49 |
| FAP4-1BBL | P1AD5195 | 51 | 52 | 53 | 54 |

Animals were controlled daily for clinical symptoms and detection of adverse effects. Tumor volume was measured by caliper, and body weight was controlled twice per week.

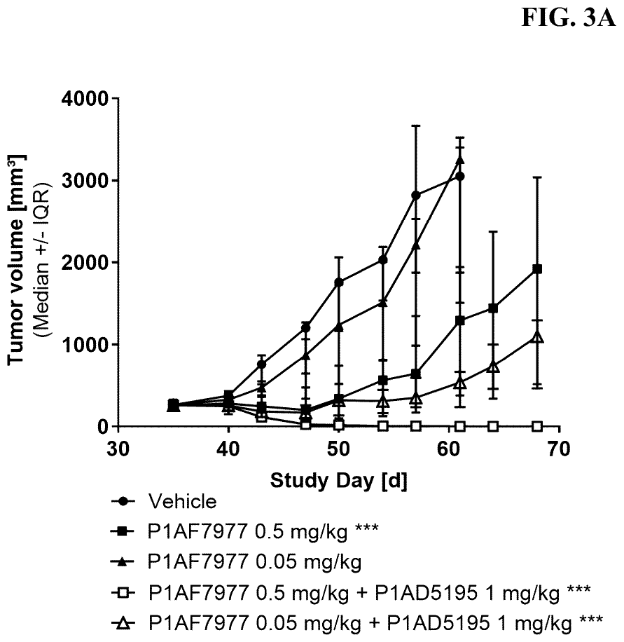

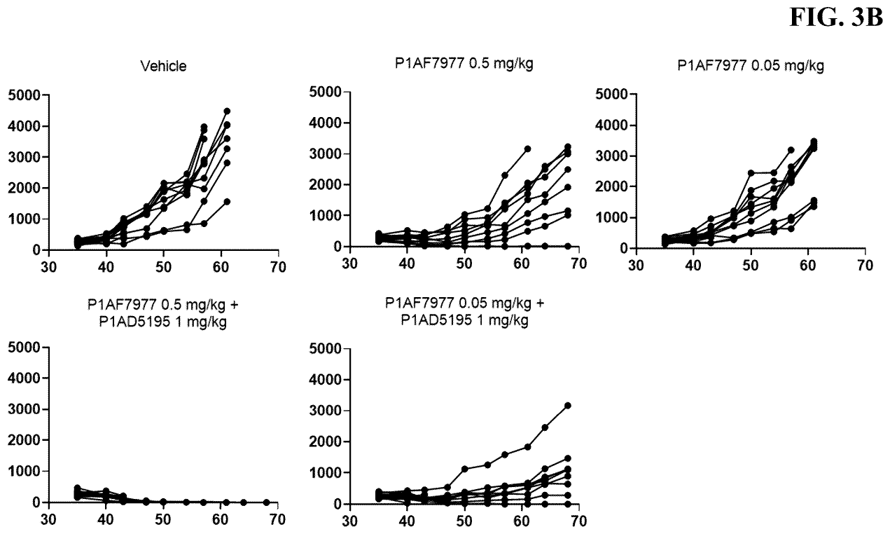

Treatment with HLA-G TCB resulted in a dose-related antitumor response in BC004 tumor-bearing animals. A small but statistically insignificant tumor growth inhibition was observed at a weekly dose of 0.05 mg/kg, but the reduction in tumor volume was much more pronounced at a weekly dose of 0.5 mg/kg (FIG. 3). Compared to HLA-G TCB monotherapy, tumor growth inhibition was clearly increased after combination of HLA-G TCB therapy with FAP4-1BBL. In the BC004 model, a dose of 0.05 mg/kg HLA-G TCB in combination with FAP4-1BBL led to significantly higher tumor volume reduction than HLA-G TCB alone at a dose of 0.05 mg/kg. The combination of 0.5 mg/kg HLA-G TCB with FAP4-1BBL resulted in all mice being tumor-free. Graphs were generated using GraphPad prism Software. A time-to-event analysis was performed using the internal tool DOPSa (based on the software R). Study groups were compared using the Log-Rank test and p-values have been corrected for multiple testing using the Bonferroni-Holm method. Significant changes compared to the control group (vehicle) with p<0.05 were depicted with asterisks (*p<0.05, **p<0.01, ***p<0.001).

To evaluate the effects of HLA-G TCB treatment as monotherapy and as combination therapy on immunoPD changes, flow cytometry analysis was performed on treatment Day 9 (“dx+9”, 2 days after the second HLA-G TCB injection). Tumors were harvested from 4 mice per group and processed to obtain a single-cell suspension. Samples were disrupted using manual scissors and the Miltenyi Gentle MACS apparatus and were then digested in an enzyme mix containing DNAseI (Catalog No. 10104159001; Sigma-Aldrich) and Collagenase D (Catalog No. 11088882001; Sigma-Aldrich). After digestion, the tissue mix was filtered through a 100- and 70-μm filter and resuspended to a single-cell suspension in buffer containing a cocktail of fluorescently labeled antibodies for detection of immune cells and tumor targets. Stained cells were analyzed on a fluorescence-activated cell sorting (FACS) Fortessa device running Diva software. Raw data were analyzed using FlowJo software and a predefined gating strategy to identify tumor-infiltrating lymphocytes.

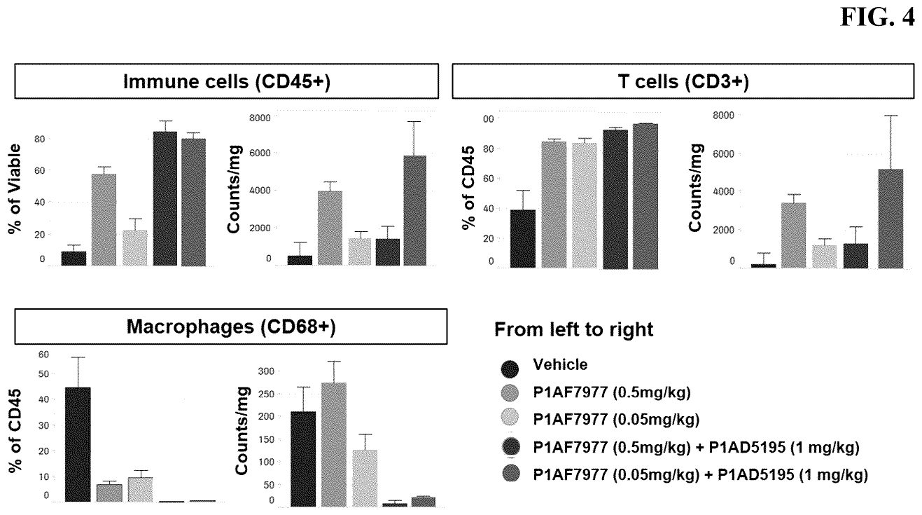

Immunopharmacodynamic (ImmunoPD) findings confirmed immune cell infiltration in general (as shown by CD45+ cells), and in particular for T cells (CD3+ cells), in the tumor in a dose-dependent manner and upregulation of the HLA-G on tumor in the different treatment groups and slightly on T cells after HLA-G TCB treatment as 0.5 mg/kg monotherapy (FIG. 4). In tumors from mice that had been treated with a combination of HLA-G TCB and FAP4-1BBL, levels of human macrophages (CD68+) were considerably lower than in tumors from mice treated with monotherapy. Since tumor associated macrophages are known to suppress T cell function, lower macrophage content in tumors may lead to decreased T cell suppression and therefore sustained T cell activation.

The results obtained provide preclinical evidence that the therapeutic response to anti-HLA-G/anti-CD3 bispecific antibody can be boosted by combination with FAP4-1BBL.

### Example 2—Antitumor Activity of HLA-G TCB Monotherapy and Combination Therapy with FAP4-1BBL in the CR5044 PDX Model in Humanized NSG Mice

To monitor tumor growth inhibition induced by either anti-HLA-G/anti-CD3 bispecific antibody alone or by a combination of anti-HLA-G/anti-CD3 bispecific antibody and FAP4-1BBL, female 3-week-old NSG mice were humanized by irradiation (140 cGy) and intravenous injection with 9×10⁴ CD34+ cord blood cells/mouse at Jackson Laboratories on day zero (=“d0”). After reaching a human immune infiltrate (human CD45) above 25% in blood, humanized NSG mice were shipped to Roche and maintained for 5 days to get accustomed to the new environment. Mice were kept under specific-pathogen-free conditions with daily cycles of 12 h light/12 h darkness according to committed guidelines (GV-Solas; Felasa; TierschG). Continuous health monitoring was carried out on a daily basis. The experimental study protocol was reviewed and approved by the local government (ROB-55.2-2532.Vet_03-20-170).

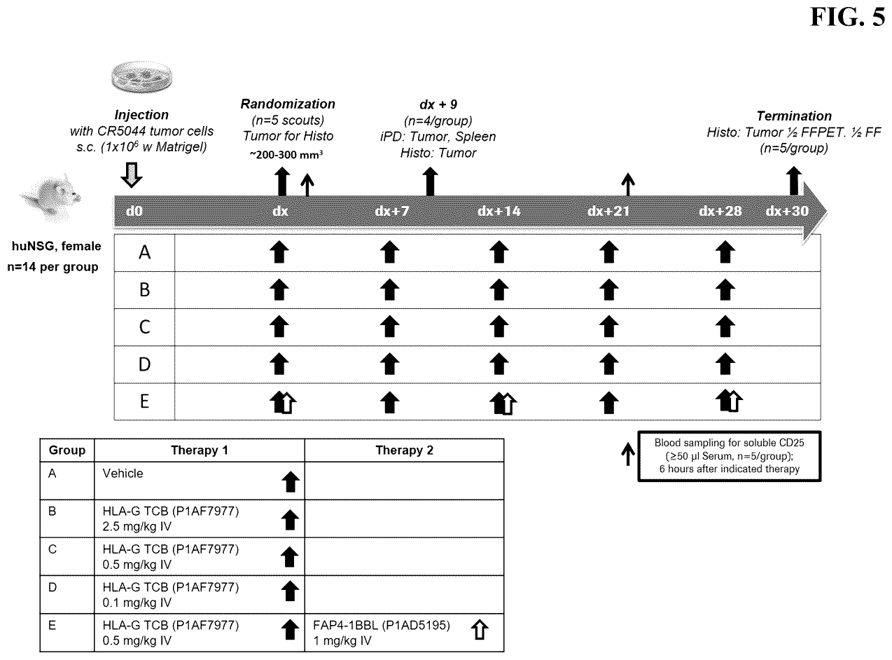

For the CR5044 PDX model, tumor cell suspensions of the human colorectal cancer PDX model CR5044 were purchased from CrownBio (San Diego, US) and mice were injected subcutaneously into the right flank with 1×10⁶ cells in 100 μL of histidine buffer and matrigel (1:1). Once tumors reached an average volume of approximately 200-300 mm³, mice were randomized into different treatment groups based on tumor volume (=“dx”). The first group of mice received histidine buffer (vehicle) as control. All molecules were prepared freshly in 20 mM Histidine, 140 mM NaCl, pH 6.0, before injection and administered intravenously (IV) at the dose and schedule indicated in FIG. 5. HLA-G TCB was injected weekly and FAP4-1BBL every 2 weeks. The administered molecules had the same sequences as described in Example 1.

Animals were controlled daily for clinical symptoms and detection of adverse effects. Tumor volume was measured by caliper, and body weight was controlled twice per week. Termination criteria for animals were visible sickness (scruffy fur, arched back, breathing problems, impaired locomotion), body weight loss (≥25% within 7 days after first treatment or ≥20% after 7 days of treatment), or tumor size (diameter ≥2 cm, volume ≥4000 mm³). Animals were sacrificed according to the termination criteria or at the end of the experiment.

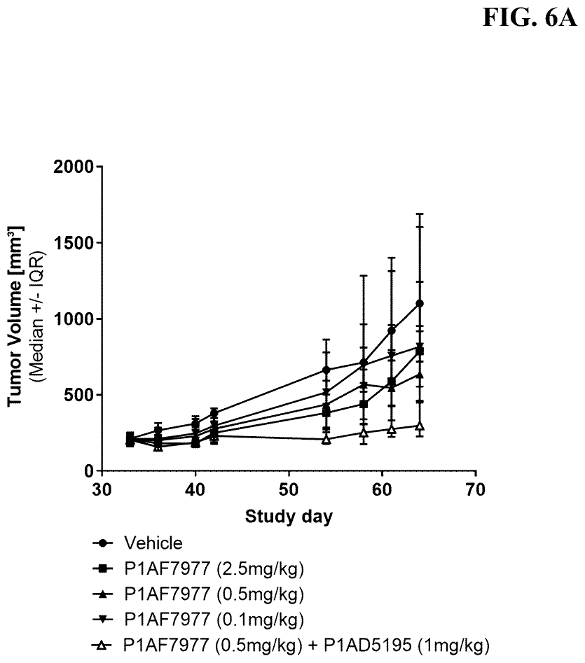

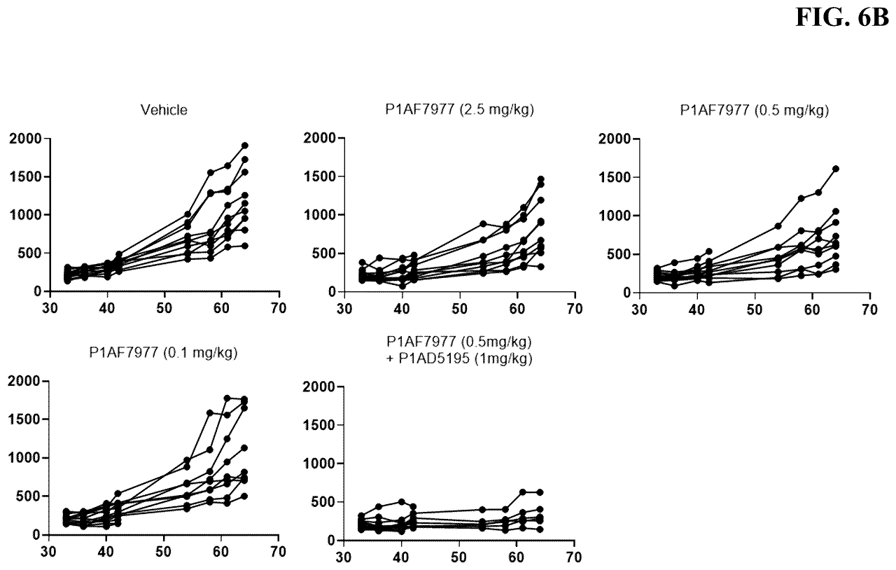

In the CR5044 PDX model, only minor antitumor activity after HLA-G TCB monotherapy was measured for treatment with 2.5 mg/kg and 0.5 mg/kg HLA-G TCB. In comparison, TGI was clearly increased after combination therapy with HLA-G TCB and FAP4-1BBL, and the response was more homogeneous compared with HLA-G TCB monotherapy. (FIG. 6). Graphs were generated using GraphPad prism Software. A time-to-event analysis was performed using the internal tool DOPSa (based on the software R). Study groups were compared using the Log-Rank test and p-values have been corrected for multiple testing using the Bonferroni-Holm method. Significant changes compared to the control group (vehicle) with p<0.05 were depicted with asterisks (*p<0.05, **p<0.01, ***p<0.001).

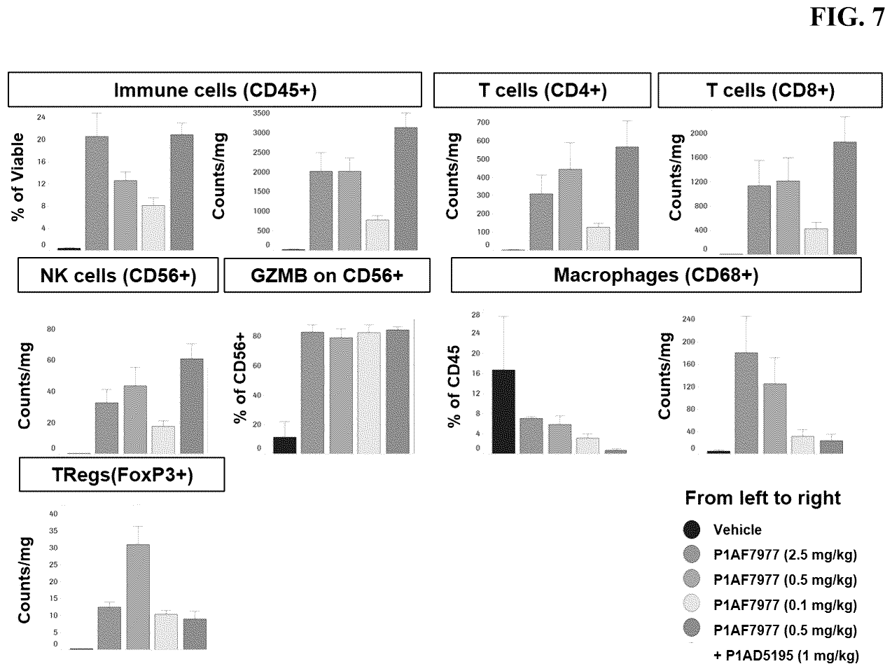

To evaluate the effects of HLA-G TCB treatment, as monotherapy and as combination therapy with FAP4-1BBL, on immunoPD changes, flow cytometry analysis was performed on treatment Day 9 (“dx+9”, 2 days after the second injection). Tumors were harvested from 4 mice/group and processed to obtain a single-cell suspension. Samples were disrupted using manual scissors and the Miltenyi Gentle MACS apparatus and were then digested in an enzyme mix containing DNAseI (Catalog No. 10104159001; Sigma-Aldrich) and Collagenase D (Catalog No. 11088882001; Sigma-Aldrich). After digestion, the tissue mix was filtered through a 100- and 70-μm filter and resuspended to a single-cell suspension in buffer containing a cocktail of fluorescently labeled antibodies for detection of immune cells and tumor targets. Stained cells were analyzed on a fluorescence-activated cell sorting (FACS) Fortessa device running Diva software. Raw data were analyzed using FlowJo software and a predefined gating strategy to identify tumor-infiltrating lymphocytes. Immunopharmacodynamics analysis confirmed immune cell infiltration in the tumor (such as CD45+, CD4+ and CD8+ T cells, TRegs and NK cells) after PIAF7977 monotherapy and combination therapy with FAP4-1BBL (FIG. 7). In tumors from mice that had been treated with a combination of HLA-G TCB and FAP4-1BBL, levels of macrophages (CD68+) were considerably lower than in tumors from mice treated with PIAF7977 monotherapy at 0.5 mg/kg, indicating a potential reduction of macrophage-mediated suppression of T cell activation.

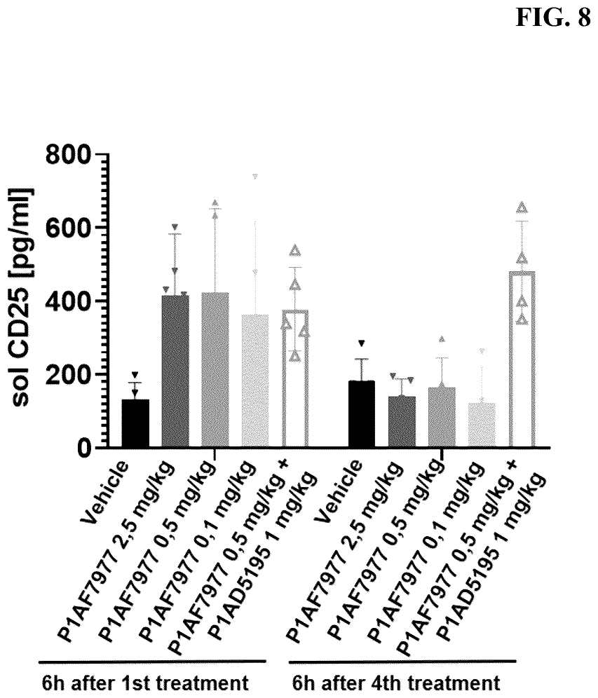

The levels of soluble CD25 were measured in 50 μL serum samples (1:5 dilution) at 2 different timepoints (6 hours after the first and fourth treatment cycle, respectively) using the human CD25/interleukin (IL)-2R alpha kit (Catalog No. LXSAHM-01; R&D Systems; Minneapolis; MN). The levels of soluble CD25 were increased in serum of CR5044 tumor-bearing mice after HLA-G TCB monotherapy and combination with PIAD5195, indicating T cell activation upon treatment (FIG. 8). In the combination treatment group, elevated CD25 levels in serum were observed for a prolonged time, i.e. until after the 4^{th} HLA-G TCB treatment cycle, indicating sustained T cell activation (FIG. 8, left).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An anti-HLA-G/anti-CD3 bispecific antibody for use in the treatment of a cancer in an individual, wherein the treatment comprises administration of the anti-HLA-G/anti-CD3 bispecific antibody in combination with a 4-1BB (CD137) agonist.

2. A 4-1BB (CD137) agonist for use in the treatment of a cancer in an individual, wherein the treatment comprises administration of the 4-1BB (CD137) agonist in combination with an anti-HLA-G/anti-CD3 bispecific antibody.

3. Use of an anti-HLA-G/anti-CD3 bispecific antibody in the manufacture of a medicament for the treatment of cancer in an individual, wherein the treatment comprises administration of the anti-HLA-G/anti-CD3 bispecific antibody in combination with a 4-1BB (CD137) agonist.

4. Use of a 4-1BB (CD137) agonist in the manufacture of a medicament for the treatment of cancer in an individual, wherein the treatment comprises administration of the 4-1BB (CD137) agonist in combination with an anti-HLA-G/anti-CD3 bispecific antibody.

5. A method for treating cancer in an individual comprising administering to the individual an anti-HLA-G/anti-CD3 bispecific antibody and a 4-1BB (CD137) agonist.

6. A kit comprising a first medicament comprising an anti-HLA-G/anti-CD3 bispecific antibody and a second medicament comprising a 4-1BB (CD137) agonist, and optionally further comprising a package insert comprising instructions for administration of the first medicament in combination with the second medicament for treating cancer in an individual.

7. The anti-HLA-G/anti-CD3 bispecific antibody for use, the 4-1BB (CD137) agonist for use, the use, the method or the kit of any one of the preceding claims, wherein the anti-HLA-G/anti-CD3 bispecific antibody comprises
(i) a first antigen-binding moiety that specifically binds to CD3 and comprises a heavy chain variable region comprising the heavy chain CDR (CDR-H) 1 of SEQ ID NO: 1, the CDR-H2 of SEQ ID NO: 2, and the CDR-H3 of SEQ ID NO: 3; and a light chain variable region comprising the light chain CDR (CDR-L) 1 of SEQ ID NO: 4, the CDR-L2 of SEQ ID NO: 5 and the CDR-L3 of SEQ ID NO: 6; and
(ii) a second antigen-binding moiety that specifically binds to HLA-G and comprises a heavy chain variable region comprising the heavy chain CDR (CDR-H) 1 of SEQ ID NO: 9, the CDR-H2 of SEQ ID NO: 10, and the CDR-H3 of SEQ ID NO: 11; and a light chain variable region comprising the light chain CDR (CDR-L) 1 of SEQ ID NO: 12, the CDR-L2 of SEQ ID NO: 13 and the CDR-L3 of SEQ ID NO: 14.

8. The anti-HLA-G/anti-CD3 bispecific antibody for use, the 4-1BB (CD137) agonist for use, the use, the method or the kit of any one of the preceding claims, wherein the anti-HLA-G/anti-CD3 bispecific antibody comprises a third antigen-binding moiety that specifically binds to HLA-G and/or an Fc domain composed of a first and a second subunit.

9. The anti-HLA-G/anti-CD3 bispecific antibody for use, the 4-1BB (CD137) agonist for use, the use, the method or the kit of any one of the preceding claims, wherein the anti-HLA-G/anti-CD3 bispecific antibody comprises
(i) a first antigen-binding moiety that specifically binds to CD3, comprising a heavy chain variable region comprising the heavy chain CDR (CDR-H) 1 of SEQ ID NO: 1, the CDR-H2 of SEQ ID NO: 2, and the CDR-H3 of SEQ ID NO: 3; and a light chain variable region comprising the light chain CDR (CDR-L) 4 of SEQ ID NO: 4, the CDR-L2 of SEQ ID NO: 5 and the CDR-L3 of SEQ ID NO: 6, wherein the first antigen-binding moiety is a crossover Fab molecule wherein either the variable or the constant regions of the Fab light chain and the Fab heavy chain are exchanged;
(ii) a second and a third antigen-binding moiety that specifically bind to HLA-G, comprising a heavy chain variable region comprising the heavy chain CDR (CDR-H) 1 of SEQ ID NO: 9, the CDR-H2 of SEQ ID NO: 10, and the CDR-H3 of SEQ ID NO: 11; and a light chain variable region comprising the light chain CDR (CDR-L) 1 of SEQ ID NO: 12, the CDR-L2 of SEQ ID NO: 13 and the CDR-L3 of SEQ ID NO: 14, wherein the second and third antigen-binding moiety are each a Fab molecule, particularly a conventional Fab molecule; and
(iii) an Fc domain composed of a first and a second subunit,
wherein the second antigen-binding moiety is fused at the C-terminus of the Fab heavy chain to the N-terminus of the Fab heavy chain of the first antigen-binding moiety, and the first antigen-binding moiety is fused at the C-terminus of the Fab heavy chain to the N-terminus of the first subunit of the Fc domain, and wherein the third antigen-binding moiety is fused at the C-terminus of the Fab heavy chain to the N-terminus of the second subunit of the Fc domain.

10. The anti-HLA-G/anti-CD3 bispecific antibody for use, the 4-1BB (CD137) agonist for use, the use, the method or the kit of any one of the preceding claims, wherein the first antigen-binding moiety of the anti-HLA-G/anti-CD3 bispecific antibody comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8, and/or the second and (where present) third antigen-binding moiety of the anti-HLA-G/anti-CD3 bispecific antibody comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

11. The anti-HLA-G/anti-CD3 bispecific antibody for use, the 4-1BB (CD137) agonist for use, the use, the method or the kit of any one of the preceding claims, wherein the first antigen-binding moiety of the anti-HLA-G/anti-CD3 bispecific antibody is a crossover Fab molecule wherein the variable regions of the Fab light chain and the Fab heavy chain are exchanged, and wherein the second and (where present) third antigen-binding moiety of the anti-HLA-G/anti-CD3 bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

12. The anti-HLA-G/anti-CD3 bispecific antibody for use, the 4-1BB (CD137) agonist for use, the use, the method or the kit of any one of the preceding claims, wherein the Fc domain of the anti-HLA-G/anti-CD3 bispecific antibody comprises a modification promoting the association of the first and the second subunit of the Fc domain, and/or the Fc domain comprises one or more amino acid substitutions that reduce binding to an Fc receptor and/or effector function.

13. The anti-HLA-G/anti-CD3 bispecific antibody for use, the 4-1BB (CD137) agonist for use, the use, the use, the method or the kit of any one of the preceding claims, wherein the anti-HLA-G/anti-CD3 bispecific antibody comprises a first polypeptide comprising an amino acid sequence that is at least about 95%, 96%, 97%, 98%, 99% or 100% identical to the amino acid sequence of SEQ ID NO: 46, a second polypeptide comprising an amino acid sequence that is at least about 95%, 96%, 97%, 98%, 99% or 100% identical to the amino acid sequence of SEQ ID NO: 47, a third polypeptide comprising an amino acid sequence that is at least about 95%, 96%, 97%, 98%, 99% or 100% identical to the amino acid sequence of SEQ ID NO: 48, and a fourth polypeptide comprising an amino acid sequence that is at least about 95%, 96%, 97%, 98%, 99% or 100% identical to the amino acid sequence of SEQ ID NO: 49.

14. The anti-HLA-G/anti-CD3 bispecific antibody for use, the 4-1BB (CD137) agonist for use, the use, the method or the kit of any one of the preceding claims, wherein the 4-1BB (CD137) agonist comprises an ectodomain of 4-1BBL or fragment thereof, particularly three ectodomains of 4-1BBL or fragments thereof.

15. The anti-HLA-G/anti-CD3 bispecific antibody for use, the 4-1BB (CD137) agonist for use, the use, the method or the kit of any one of the preceding claims, wherein the ectodomain of 4-1BBL or fragment thereof comprises an amino acid sequence that is at least about 95%, 96%, 97%, 98%, 99% or 100% identical to an amino acid sequence selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32, particularly the amino acid sequence of SEQ ID NO: 29.

16. The anti-HLA-G/anti-CD3 bispecific antibody for use, the 4-1BB (CD137) agonist for use, the use, the method or the kit of any one of the preceding claims, wherein the 4-1BB (CD137) agonist comprises an antigen-binding moiety that specifically binds to a tumor-associated antigen, particularly Fibroblast Activation Protein (FAP).

17. The anti-HLA-G/anti-CD3 bispecific antibody for use, the 4-1BB (CD137) agonist for use, the use, the method or the kit of any one of the preceding claims, wherein the antigen-binding moiety that specifically binds to FAP comprises a heavy chain variable region (VH) comprising the heavy chain CDR (CDR-H) 1 of SEQ ID NO: 17, the CDR-H2 of SEQ ID NO: 18, and the CDR-H3 of SEQ ID NO: 19; and a light chain variable region comprising the light chain CDR (CDR-L) 1 of SEQ ID NO: 20, the CDR-L2 of SEQ ID NO: 21 and the CDR-L3 of SEQ ID NO: 22.

18. The anti-HLA-G/anti-CD3 bispecific antibody for use, the 4-1BB (CD137) agonist for use, the use, the method or the kit of any one of the preceding claims, wherein the antigen-binding moiety that specifically binds to FAP comprises a heavy chain variable region sequence that is at least about 95%, 96%, 97%, 98%, 99% or 100% identical to the amino acid sequence of SEQ ID NO: 23 and a light chain variable region sequence that is at least about 95%, 96%, 97%, 98%, 99% or 100% identical to the amino acid sequence of SEQ ID NO: 24.

19. The anti-HLA-G/anti-CD3 bispecific antibody for use, the 4-1BB (CD137) agonist for use, the use, the method or the kit of any one of the preceding claims, wherein the 4-1BB (CD137) agonist comprises an Fc domain composed of a first and a second subunit.

20. The anti-HLA-G/anti-CD3 bispecific antibody for use, the 4-1BB (CD137) agonist for use, the use, the method or the kit of any one of the preceding claims, wherein the Fc domain of the 4-1BB (CD137) agonist comprises a modification promoting the association of the first and the second subunit of the Fc domain, and/or the Fc domain comprises one or more amino acid substitutions that reduce binding to an Fc receptor and/or effector function.

21. The anti-HLA-G/anti-CD3 bispecific antibody for use, the 4-1BB (CD137) agonist for use, the use, the method or the kit of any one of the preceding claims, wherein the 4-1BB (CD137) agonist is an antigen-binding molecule comprising
(i) a first, a second and a third ectodomain of 4-1BBL or a fragment thereof;
(ii) an antigen-binding moiety that specifically binds to FAP, wherein the antigen-binding moiety is a Fab molecule;
(iii) an Fc domain composed of a first and a second subunit;
(iv) a CL domain and a CH1 domain;
wherein the antigen-binding molecule is composed of
(a) a first polypeptide, comprising the (a1) first ectodomain of 4-1BBL or fragment thereof, fused at its C-terminus to the N-terminus of the second ectodomain of 4-1BBL or fragment thereof, (a2) the second ectodomain of 4-1BBL or fragment thereof, fused at its C-terminus to the N-terminus of the CL domain, (a3) the CL domain, fused at its C-terminus to the N-terminus of one of the subunits (e.g. the first subunit) of the Fc domain, and (a4) one of the subunits (e.g. the first subunit) of the Fc domain;
(b) a second polypeptide, comprising (b1) the third ectodomain of 4-1BBL or fragment thereof, fused at its C-terminus to the N-terminus of the CH1 domain, and (b2) the CH1 domain;
(c) a third polypeptide, comprising (c1) the heavy chain of the Fab molecule, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising the light chain of the Fab molecule.

22. The anti-HLA-G/anti-CD3 bispecific antibody for use, the 4-1BB (CD137) agonist for use, the use, the use, the method or the kit of claim 21, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

23. The anti-HLA-G/anti-CD3 bispecific antibody for use, the 4-1BB (CD137) agonist for use, the use, the use, the method or the kit of claim 21 or claim 22, wherein the first polypeptide of the 4-1BB (CD137) agonist comprises an amino acid sequence that is at least about 95%, 96%, 97%, 98%, 99% or 100% identical to the amino acid sequence of SEQ ID NO: 51, the second polypeptide of the 4-1BB (CD137) agonist comprises an amino acid sequence that is at least about 95%, 96%, 97%, 98%, 99% or 100% identical to the amino acid sequence of SEQ ID NO: 52, the third polypeptide of the 4-1BB (CD137) agonist comprises an amino acid sequence that is at least about 95%, 96%, 97%, 98%, 99% or 100% identical to the amino acid sequence of SEQ ID NO: 53, and the fourth polypeptide of the 4-1BB (CD137) agonist comprises an amino acid sequence that is at least about 95%, 96%, 97%, 98%, 99% or 100% identical to the amino acid sequence of SEQ ID NO: 54.

24. The anti-HLA-G/anti-CD3 bispecific antibody for use, the 4-1BB (CD137) agonist for use, the use, the use, the method or the kit of any one of claim 1 to claim 13, wherein the 4-1BB (CD137) agonist is an anti-4-1BB antibody, particularly an anti-FAP/anti-4-1BB bispecific antibody.

25. The anti-HLA-G/anti-CD3 bispecific antibody for use, the 4-1BB (CD137) agonist for use, the use, the use, the method or the kit of any one of the preceding claims, wherein the cancer is a HLA-G-positive cancer.

26. The anti-HLA-G/anti-CD3 bispecific antibody for use, the 4-1BB (CD137) agonist for use, the use, the use, the method or the kit of any one of the preceding claims, wherein the cancer is a cancer selected from the group consisting of lung cancer, head and neck cancer, bladder cancer, esophageal cancer, skin cancer, soft tissue cancer, gastric cancer, cervical cancer and ovarian cancer.

27. The invention as described hereinbefore.


SEQUENCE LISTING (64 sequences)

SEQ ID NO: 1 - protein, 5 aa, synthetic construct
SYAMN

SEQ ID NO: 2 - protein, 19 aa, synthetic construct
RIRSKYNNYATYYADSVKG

SEQ ID NO: 3 - protein, 14 aa, synthetic construct
ASNFPASYVSYFAY

SEQ ID NO: 4 - protein, 14 aa, synthetic construct
GSSTGAVTTSNYAN

SEQ ID NO: 5 - protein, 7 aa, synthetic construct
GTNKRAP

SEQ ID NO: 6 - protein, 9 aa, synthetic construct
ALWYSNLWV

SEQ ID NO: 7 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 8 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 9 - protein, 7 aa, synthetic construct
SNRAAWN

SEQ ID NO: 10 - protein, 18 aa, synthetic construct
RTYYRSKWYNDYAVSVQG

SEQ ID NO: 11 - protein, 9 aa, synthetic construct
VRAVAPFDY

SEQ ID NO: 12 - protein, 17 aa, synthetic construct
KSSQSVLNPSNNKNNLA

SEQ ID NO: 13 - protein, 7 aa, synthetic construct
WASTRES

SEQ ID NO: 14 - protein, 9 aa, synthetic construct
QQYYRTPWT

SEQ ID NO: 15 - protein, 121 aa, synthetic construct
QVQLQQSGPGLLKPSQTLSLTCAISGDSVSSNRAAWNWIRQSPSRGLEWLGRTYYRSKWYNDYAVSVQGRITLIPDTSKNQFSLRLNSVTPEDTAVYYCASVRAVAPFDYWGQGVLVTVSS

SEQ ID NO: 16 - protein, 113 aa, synthetic construct
DIVMTQSPDSLAVSLGERATINCKSSQSVLNPSNNKNNLAWYQQQPGQPPKLLIYWASTRESGVPDRFSGSGSGTDFTLTISSLQAEDVAVYFCQQYYRTPWTFGQGTKVEIK

SEQ ID NO: 17 - protein, 5 aa, synthetic construct
SYAMS

SEQ ID NO: 18 - protein, 17 aa, synthetic construct
AIIGSGASTYYADSVKG

SEQ ID NO: 19 - protein, 8 aa, synthetic construct
GWFGGFNY

SEQ ID NO: 20 - protein, 12 aa, synthetic construct
RASQSVTSSYLA

SEQ ID NO: 21 - protein, 7 aa, synthetic construct
VGSRRAT

SEQ ID NO: 22 - protein, 9 aa, synthetic construct
QQGIMLPPT

SEQ ID NO: 23 - protein, 117 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 24 - protein, 108 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 25 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 26 - protein, 170 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 27 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 28 - protein, 203 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 29 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 30 - protein, 164 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 31 - protein, 169 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 32 - protein, 197 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 33 - protein, 378 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 34 - protein, 350 aa, synthetic construct
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 35 - protein, 360 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 36 - protein, 416 aa, synthetic construct
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 37 - protein, 366 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 38 - protein, 338 aa, synthetic construct
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 39 - protein, 348 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 40 - protein, 404 aa, synthetic construct
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 41 - protein, 314 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 42 - protein, 440 aa, Homo sapiens
RIIPRHLQLGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWGSGLNDIFEAQKIEWHEHHHHHH

SEQ ID NO: 43 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 44 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 45 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 46 - protein, 220 aa, synthetic construct
DIVMTQSPDSLAVSLGERATINCKSSQSVLNPSNNKNNLAWYQQQPGQPPKLLIYWASTRESGVPDRFSGSGSGTDFTLTISSLQAEDVAVYFCQQYYRTPWTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 451 aa, synthetic construct
QVQLQQSGPGLLKPSQTLSLTCAISGDSVSSNRAAWNWIRQSPSRGLEWLGRTYYRSKWYNDYAVSVQGRITLIPDTSKNQFSLRLNSVTPEDTAVYYCASVRAVAPFDYWGQGVLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 232 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 676 aa, synthetic construct
QVQLQQSGPGLLKPSQTLSLTCAISGDSVSSNRAAWNWIRQSPSRGLEWLGRTYYRSKWYNDYAVSVQGRITLIPDTSKNQFSLRLNSVTPEDTAVYYCASVRAVAPFDYWGQGVLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGGQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 366 aa, synthetic construct; identical to SEQ ID NO: 37

SEQ ID NO: 51 - protein, 708 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 52 - protein, 291 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 53 - protein, 445 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 54 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 56 - protein, 5 aa, synthetic construct
GGGGS

SEQ ID NO: 57 - protein, 10 aa, synthetic construct
GGGGSGGGGS

SEQ ID NO: 58 - protein, 15 aa, synthetic construct
GGGGSGGGGSGGGGS

SEQ ID NO: 59 - protein, 20 aa, synthetic construct
GGGGSGGGGSGGGGSGGGGS

SEQ ID NO: 60 - protein, 10 aa, synthetic construct
GGGGSGGGGG

SEQ ID NO: 61 - protein, 4 aa, synthetic construct
GSGS

SEQ ID NO: 62 - protein, 5 aa, synthetic construct
  REGION 1..5: note = The entire sequence of amino acids 1-5 can be repeated one or more times.
GGGGS

SEQ ID NO: 63 - protein, 5 aa, synthetic construct
  REGION 1..5: note = The entire sequence of amino acids 1-5 can be repeated one or more times.
SGGGG

SEQ ID NO: 64 - protein, 9 aa, synthetic construct
  REGION 5..9: note = The entire sequence of amino acids 5-9 can be repeated one or more times.
GGGGSGGGG